(19) United States
(12) Patent Application Publication
(10) Pub. No.: US 2021/0403498 A1
(43) Pub. Date: Dec. 30, 2021

(54) PHYTOCHEMICAL-FRUCTOOLIGOSACCHARIDE CONJUGATE, METHOD FOR PRODUCING SAME, AND USE THEREOF

(71) Applicant: EVERGREEN BIO CO., LTD., Yongin-si, Gyeonggi-do (KR)

(72) Inventors: Joo Won SUH, Yongin-si (KR); Eldin Maliyakkal JOHNSON, Tamil Nadu (IN); Han Ki LEE, Yongin-si (KR)

(73) Assignee: EVERGREEN BIO CO., LTD., Yongin-si, Gyeonggi-do (KR)

(21) Appl. No.: 17/289,862

(22) PCT Filed: Oct. 30, 2019
(86) PCT No.: PCT/KR2019/014489

§ 371 Date: Apr. 29, 2021

(30) Foreign Application Priority Data
Oct. 30, 2018 (KR) 10-2018-0130990

(51) Int. Cl. C07H 3/06; C07H 1/00; B01J 31/02

(52) U.S. Cl. CPC C07H 3/06; B01J 31/02; C07H 1/00

(57) ABSTRACT

The present invention provides a novel phytochemical-fructooligosaccharide conjugate having a structure in which a phytochemical in the form of phenolic acid is conjugated to a portion of saccharides constituting a fructooligosaccharide backbone. Compared to corresponding phenolic acid phytochemicals, the novel phytochemical-fructooligosaccharide conjugate according to the present invention is hardly decomposed under oral-gastrointestinal transit conditions such that most of the phytochemical-fructooligosaccharide conjugate may reach the large intestine. Thus, the novel phytochemical-fructooligosaccharide conjugate has good bioavailability in the large intestine when orally administered. In particular, a ferulic acid-fructooligosaccharide conjugate according to the present invention has an excellent colorectal cancer cell-selective killing effect as compared to commercial colorectal cancer treatment drugs, and may thus be used as a food or pharmaceutical material useful for preventing, alleviating, or treating colorectal cancer.

### TECHNICAL FIELD

The present disclosure relates to a phytochemical-fructooligosaccharide conjugate, and more specifically, a novel substance formed via side chain bonding of phytochemical in a phenolic acid to some of saccharides constituting a main chain of fructooligosaccharide, a method for producing the same, and various uses thereof based on physiological activity thereof.

### BACKGROUND ART

It is reported that colorectal cancer (CRC) has the third highest cancer mortality in the world, and one million or more people have the colorectal cancer each year. While the genetic risk of the onset of colorectal cancer is about 10%, surprisingly, the remaining 90% is due to an unhealthy lifestyle and eating habits. Most drugs and compounds developed to treat colorectal cancer are synthetics or biosimilars, which cause unintended problems including serious side effects such as complications or low quality life in patients during or after treatment. The survival percentage of CRC patients is one of the biggest challenges in the science field because most CRC patients are exacerbated by serious complications when receiving traditional treatments such as radiation therapy, chemotherapy and other interventional therapies. Further, an appropriate treatment plan with a low incidence of recurrence after the improvement during treatment should be developed. Despite the best efforts to eliminate cancer using currently available treatment plans or systems, the cancer may recur frequently. Therefore, there is a high demand for cancer treatments with little or no side effects and a high recovery rate for patients. Further, oxidative stress and related diseases are also known to be responsible for various health problems and complications.

In the meantime, phytochemical derived from a food source is a chemical substance contained in plants, and plays a role in preventing the growth of competing plants or protecting the body from various microorganisms or pests. Further, when phytochemical enters the human body, it acts as an antioxidants or inhabits cell damage, thereby maintaining health. Representative examples thereof include aspirin extracted from willow bark, quinine as a special anti-malarial drug, flavonoids and carotenoids that inhibit the production of carcinogens. Among the phytochemicals, phenolic compounds are known to have various health benefits, but their beneficial characteristics are alleviated because of their low solubility and bioavailability. Further, most of the phenolic compounds are absorbed in the small intestine and metabolized in the liver via conjugation reactions such as glucuronidation, sulphation, and methylation. Thus, only very low concentrations of free aglycone are biologically available in plasma. For example, ferulic acid as one of the phytochemicals in the form of phenolic acid is an antioxidant and reacts with free radicals such as reactive oxygen species (ROS) to relieve oxidative stress, thereby reducing and ameliorating DNA damage, cancer, and cell aging. However, since ferulic acid administered orally is mainly decomposed and absorbed in the stomach or small intestine, and metabolized in the liver, only a very small amount thereof reaches the large intestine. [See Cesare Mancuso et al.: Ferulic acid: Pharmacological and toxicological aspects. Food and Chemical Toxicology 65 (2014) 185-195]. Therefore, there is a need to increase the bioavailability of phenolic compounds derived from botanical sources and having physiological activity in plasma. Further, in order to improve the colorectal cancer treatment effect of the phenolic compound, a technology for modifying a phenolic compound is required such that the compound is not decomposed in the oral cavity, stomach, or small intestine and is targeted and delivered to a large intestine. Prior documents related to the phenolic compound modification technology include Japanese Patent No. 5900792, Japanese Unexamined Patent Publication No. 2006-89437, and the like.

### DISCLOSURE

### Technical Problem

The present disclosure is derived from the prior technical background. A purpose of the present disclosure is to provide a novel phytochemical-fructooligosaccharide conjugate that has the same or improved physiological activity as that of phytochemical in the form of phenolic acid, and at the same time, is hardly decomposed in the oral, gastric and small intestine environments when administered orally, and may reach large intestines.

Further, a purpose of the present disclosure is to provide a method for producing a novel phytochemical-fructooligosaccharide conjugate.

Further, a purpose of the present disclosure is to provide various pharmaceutical uses of the novel phytochemical-fructooligosaccharide conjugates, uses thereof for health functional foods, or uses thereof for cosmetics, based on the physiological activity thereof.

### Technical Solution

The inventors of the present disclosure have synthesized a novel phytochemical-fructooligosaccharide conjugate by binding phytochemical in the form of phenolic acid to some of saccharides constituting the main chain of fructooligosaccharide. Further, the inventors of the present disclosure have identified that the synthesized novel phytochemical-fructooligosaccharide conjugate is hardly decomposed in the oral-gastrointestinal tract passage condition compared to the corresponding phenolic acid-type phytochemical, and most of them reach large intestines, and at the same time, has excellent selective killing effect of the cancer cells, and especially the colorectal cancer treatment effect thereof is very excellent. Thus, the present disclosure has been completed.

In order to achieve one purpose of the present disclosure, an example of the present disclosure provides a phytochemical-fructooligosaccharide conjugate represented by a following chemical formula I or chemical formula II.

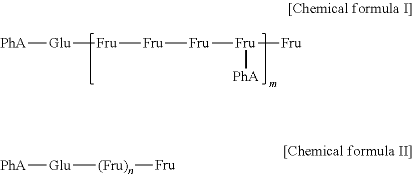

In chemical formula I and chemical formula II, ‘PhA’ represents phytochemical in the form of phenolic acid, ‘Glu’ represents glucose, and ‘Fru’ represents fructose. Further, in the chemical formula I and chemical formula II, ‘PhA’ and ‘Glu’ are linked to each other via an ester bond, ‘Glu’ and ‘Fru’ are linked to each other via a glycosidic bond, and ‘Fru’ and ‘Fru’ is linked to each other via a glycosidic bond, and ‘Fru’ and ‘PhA’ are linked to each other via an ester bond. Further, in the chemical formula I, m is the number of repeating units, each composed of four ‘Fru’s connected to each other by a glycosidic bond and one ‘PhA’ connected to ‘Fru’ via an ester bond, and is selected from an integer of 1 to 14. Further, in the chemical formula II, n is the number of ‘Fru’s linked to each other via a glycosidic bond, and is selected from an integer of 1 to 59.

In order to achieve one purpose of the present disclosure, an example of the present disclosure provides a pharmaceutically acceptable salt of the novel phytochemical-fructooligosaccharide conjugate described above.

In order to achieve the purpose of the present disclosure, an example of the present disclosure provides a phytochemical-fructooligosaccharide conjugate producing method comprising (a) adding, dissolving, and heating a phytochemical in the form of phenolic acid and a catalyst for esterification reaction to a reaction solvent to perform an activation reaction of the phytochemical and obtaining a first reaction mixture containing a phytochemical in an activated form; and (b) adding fructooligosaccharide represented by a following general structural formula to the first reaction mixture, followed by heating in an inert gas atmosphere to perform an esterification reaction between fructooligosaccharide and phytochemical to obtain a second reaction mixture containing the phytochemical-fructooligosaccharide conjugate.

[General Structural Formula of Fructooligosaccharide]

Glu-(Fru)ₖ

In the general structural formula of fructooligosaccharide, ‘Glu’ denotes glucose, ‘Fru’ denotes fructose, and k denotes the number of fructoses linked to each other via a glycosidic bond, and is selected from an integer of 2 to 60.

In order to achieve one purpose of the present disclosure, an example of the present disclosure provides an anticancer composition comprising the above-described novel phytochemical-fructooligosaccharide conjugate or a pharmaceutically acceptable salt thereof as an active ingredient.

In order to achieve the purpose of the present disclosure, an example of the present disclosure provides a pharmaceutical composition for the prevention or treatment of colorectal cancer, the composition comprising the above-described novel phytochemical-fructooligosaccharide conjugate or a pharmaceutically acceptable salt thereof as an active ingredient.

In order to achieve one purpose of the present disclosure, an example of the present disclosure provides a composition for inhibiting colorectal cancer metastasis, the composition comprising the above-described novel phytochemical-fructooligosaccharide conjugate or a pharmaceutically acceptable salt thereof as an active ingredient.

In order to achieve one purpose of the present disclosure, an example of the present disclosure provides an antioxidant composition comprising the above-described novel phytochemical-fructooligosaccharide conjugate or a pharmaceutically acceptable salt thereof as an active ingredient.

### Advantageous Effects

The novel phytochemical-fructooligosaccharide conjugate in accordance with the present disclosure has a structure in which phytochemical in the form of phenolic acid is bound to some of saccharides constituting the main chain of the fructooligosaccharide. The novel phytochemical-fructooligosaccharide conjugate according to the present disclosure has excellent bioavailability in large intestine when administered orally because it is hardly decomposed in oral-gastrointestinal tract passage conditions and most thereof may reach the large intestine compared to the corresponding phenolic acid phytochemical. Further, the novel phytochemical-fructooligosaccharide conjugate according to the present disclosure is safe for humans because it is composed of natural substances derived from food sources. In particular, a ferulic acid-fructooligosaccharide conjugate according to the present disclosure has excellent selective killing effect of the colorectal cancer cells compared to commercial colorectal cancer treatment drugs, and thus may be used as a food and medicine material useful for preventing, amelioration, or treating of the colorectal cancer. Further, the novel phytochemical-fructooligosaccharide conjugate according to the present disclosure has excellent antioxidant activity, and thus may be used as a food or pharmaceutical material or cosmetic material useful for anti-aging.

### DESCRIPTION OF DRAWINGS

FIG. 1 shows the spectral results of ferulic acid (FA), fructooligosaccharide (FOS) and FA-FOS I as analyzed by FTIR spectroscopy (Fourier-transform infrared spectroscopy).

FIG. 2 is an image result of FA-FOS I as analyzed by SEM (Scanning Electron Microscopy).

FIG. 3 shows the spectral results of ferulic acid (FA), fructooligosaccharide (FOS) and FA-FOS II as analyzed by FTIR spectroscopy (Fourier-transform infrared spectroscopy).

FIG. 4 shows the results of HPLC analysis of the decomposition behavior when FA-FOS II was cultured in a large intestine environment condition simulated by human large intestine microbiota.

FIG. 5 shows the result of observation of cancer cell death-inducing progress using a fluorescence microscope after staining with Annexin V and Propidium iodide when a HT-29 cell line and Lovo cell line are treated with oxaliplatin, FA-FOS I, FA-FOS II and ferulic acid (FA).

FIG. 6 shows the result of observation of cancer cell death-inducing progress using a fluorescence microscope after TUNEL assay (terminal deoxynucleotidyl transferase-dUTP nick end labeling) and additional Hoechst 33342 staining when HT-29 cell line and Lovo cell line are treated with FA-FOS I and FA-FOS II.

FIG. 7 schematically shows the experimental process to evaluate the anticancer activity of ferulic acid-fructooligosaccharide conjugate using a mouse model in which colon carcinoma is induced using AOM-DSS.

FIG. 8 shows the experimental results of evaluating the anticancer activity of ferulic acid-fructooligosaccharide conjugate using a mouse model in which colon carcinoma was induced using AOM-DSS. (a) the total number of tumor lesions in the colon, (b) colon index, (c) the body weight of the mouse at the end of the drug treatment and (d) the weight gain of the mouse during the treatment regimen for 4 weeks.

FIG. 9 shows the results of immunological parameters among experimental results that evaluated the anticancer activity of ferulic acid-fructooligosaccharide conjugate using a mouse model in which colon carcinoma is induced using AOM-DSS.

FIG. 10 shows the pharmacokinetic release profile of ferulic acid (FA) released from FA-FOS II in rat plasma.

FIG. 11 shows the results of experiments evaluating the anticancer activity of ferulic acid-fructooligosaccharide conjugate FA-FOS II using the HT-29 tumor bearing xenograft mouse model. (a) Tumor volume after 4 weeks of treatment, (b) Tumor growth rate, (c) tumor weight after 4 weeks of treatment, (d) body weight after 4 weeks of treatment, (e) daily weight gain during the course of treatment, and (f) weight increase profile during treatment.

FIG. 12 shows the results of measuring the antioxidant activity of FA-FOS I using ABTS[2,2′-azino-bis(3-ethylbenzothiazoline-6-sulphonic acid)] cation radical scavenging ability assay.

### DETAILED DESCRIPTION OF EMBODIMENT

Hereinafter, terms used in the present disclosure will be described.

As used in the present disclosure, the terms “pharmaceutically acceptable” and “food acceptable” mean not significantly stimulating the organism and not inhibiting the biological activity and properties of the administered active substance.

The term “colorectal cancer” as used in the present disclosure refers to a malignant tumor of the appendix, colon or rectum.

The term “prevention” as used in the present disclosure refers to any action that suppresses or delays the progression of a symptom of a particular disease by administering the composition according to the present disclosure.

The term “treatment” as used in the present disclosure refers to any action that ameliorates or beneficially alters the symptoms of a particular disease by administering the composition according to the present disclosure.

As used in the present disclosure, the term “amelioration” refers to any action that reduces at least a parameter related to the condition being treated, for example, the degree of a symptom.

As used in the present disclosure, the term “administration” means providing a subject with a given composition according to the present disclosure in any suitable way. In this connection, the subject refers to all animals, such as humans, monkeys, dogs, goats, pigs, or rats, with diseases whose symptoms may be reduced by administering the composition according to the present disclosure thereto.

As used in the present disclosure, the term “pharmaceutically effective amount” means an amount sufficient to treat a disease with a reasonable benefit or risk ratio applicable to medical treatment, and may be determined according to factors including the type of disease, severity thereof, activity of the drug, sensitivity to the drug, administration time, route of administration and rate of excretion, treatment period, drugs as used concurrently, and other factors well known in the medical field.

Hereinafter, the present disclosure will be described in detail.

One aspect of the present disclosure relates to a novel phytochemical-fructooligosaccharide conjugate that has the same or improved physiological activity as that of the phytochemical, and at the same time, is hardly decomposed in the oral, gastric and small intestine environments when administered orally, and may reach large intestines. The novel phytochemical-fructooligosaccharide conjugate according to an example of the present disclosure includes a fructooligosaccharide main chain represented by a following general structural formula; a phytochemical in the form of phenolic acid linked to glucose present at one end of the main chain via an ester bond; and a phytochemical in the form of phenolic acid selectively linked to fructose present at the main chain via an ester bond.

[General Structural Formula of Fructooligosaccharide Main Chain]

Glu-(Fru)ⱼ-Fru

In the general structural formula of the fructooligosaccharide main chain, ‘Glu’ represents glucose, ‘Fru’ represents fructose, and j is the number of fructoses linked to each other via a glycosidic bond and is selected from an integer of 1 to 59. In the general structural formula of the fructooligosaccharide main chain, j is preferably selected from an integer of 4 to 48, and is more preferably selected from an integer of 8 to 40, in consideration of the indigestibility of fructooligosaccharide or the ease in producing a phytochemical-fructooligosaccharide conjugate.

A type of the phytochemical in the form of phenolic acid which is a constituent of the novel phytochemical-fructooligosaccharide conjugate according to an example of the present disclosure is not limited to a specific type as long as it exhibits physiological activity such that it may be used in medicine or health functional food. For example, the phytochemical in the form of phenolic acid may be selected from ferulic acid, caffeic acid, cinnamic acid, chlorogenic acid, coumarin, sinapinic acid, cichoric acid, diferulic acid, coumaric acid, salicylic acid, 3-hydroxybenzoic acid, 4-hydroxybenzoic acid, vanillic acid, gallic acid, or ellagic acid.

Further, in the novel phytochemical-fructooligosaccharide conjugate according to an example of the present disclosure, the phytochemical is essentially connected to the main chain preferably via an ester bond between the hydroxyl group at the glucose carbon 6 of the main chain and the carboxyl group present in the phytochemical. Further, in the novel phytochemical-fructooligosaccharide conjugate according to an example of the present disclosure, the phytochemical is selectively connected to the main chain preferably via an ester bond between the hydroxyl group at the fructose carbon 6 of the main chain and the carboxyl group present in the phytochemical.

Further, a novel phytochemical-fructooligosaccharide conjugate according to a preferred example of the present disclosure is selected from compounds represented by the following chemical formula I or chemical formula II.

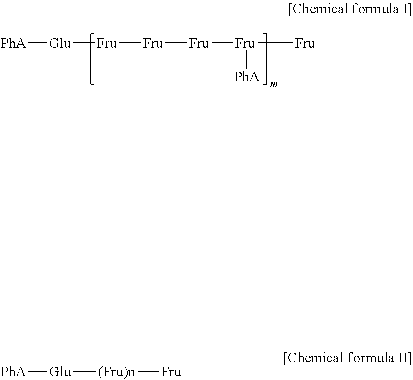

In chemical formula I and chemical formula II, ‘PhA’ represents phytochemical in the form of phenolic acid, ‘Glu’ represents glucose, and ‘Fru’ represents fructose. Further, in the chemical formula I and chemical formula II, ‘PhA’ and ‘Glu’ are connected to each other via an ester bond, ‘Glu’ and ‘Fru’ are connected to each other via a glycosidic bond, ‘Fru’ and ‘Fru’ are connected to each other via a glycosidic bond, and ‘Fru’ and ‘PhA’ are connected to each other via an ester bond. Further, in the chemical formula I, m is the number of repeating units, each composed of four ‘Fru’s connected to each other via a glycosidic bond and one ‘PhA’ connected to ‘Fru’ via an ester bond, and is selected from an integer of 1 to 14, and is preferably selected from an integer of 2 to 13, more preferably selected from an integer of 4 to 12. Further, in the chemical formula II, n is the number of ‘Fru’s connected to each other via a glycosidic bond, and is selected from an integer of 1 to 59, preferably selected from an integer of 2 to 40, more preferably, an integer of 4 to 20.

Further, a novel phytochemical-fructooligosaccharide conjugate according to a more preferred example of the present disclosure is selected from ferulic acid-fructooligosaccharide conjugates represented by a following chemical formula III or chemical formula IV. In the novel phytochemical-fructooligosaccharide conjugate according to the more preferable example of the present disclosure, the phytochemical in the form of phenolic acid is ferulic acid. The novel phytochemical-fructooligosaccharide conjugate according to the more preferred example of the present disclosure has an equivalent or improved anticancer activity (especially, anti-colorectal cancer activity) compared to ferulic acid, and at the same time, has excellent bioavailability in large intestines because it hardly decomposes in the oral, the gastric, and the small intestine environment when being administered orally and most thereof reaches the large intestine.

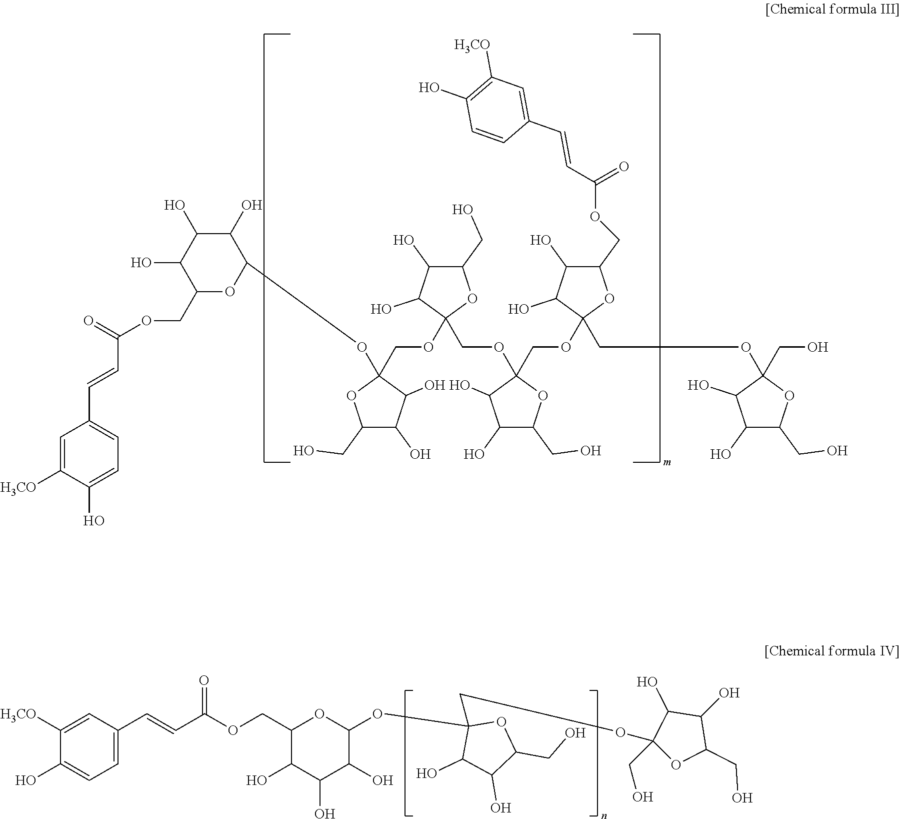

In the chemical formula III, m is the number of repeating units, each composed of four fructoses connected to each other via a glycosidic bond and one ferulic acid linked to fructose via an ester bond, and is selected from an integer of 1 to 14, preferably, selected from an integer of 2 to 13, and more preferably selected from an integer of 4 to 12. Further, in the chemical formula IV, n is the number of fructoses connected to each other via a glycosidic bond, and is selected from an integer of 1 to 59, preferably selected from an integer of 2 to 40, and more preferably selected from an integer of 4 to 20.

Further, the novel phytochemical-fructooligosaccharide conjugate according to the most preferable example of the present disclosure is composed of or contains a ferulic acid-fructooligosaccharide conjugate represented by a following chemical formula V as a main component. The inventors of the present disclosure named the ferulic acid-fructooligosaccharide conjugate containing the compound represented by the following chemical formula V as the main component as ‘FA-FOS I’. Based on the analysis of physical properties thereof, FA-FOS I is insoluble in water and forms a spherical particle having a micelle structure in an aquatic environment. Further, the ferulic acid-fructooligosaccharide conjugate FA-FOS I hardly decomposes in oral, gastric and small intestine environments and most thereof reaches the large intestine and passes through mucus in a large intestine environment and adheres to cancer cells in acidic conditions and induces death of the cancer cells. In particular, the ferulic acid-fructooligosaccharide conjugate FA-FOS I is very useful in inhibiting metastasis of colorectal cancer because it may selectively kill only colorectal cancer cells capable of metastasis.

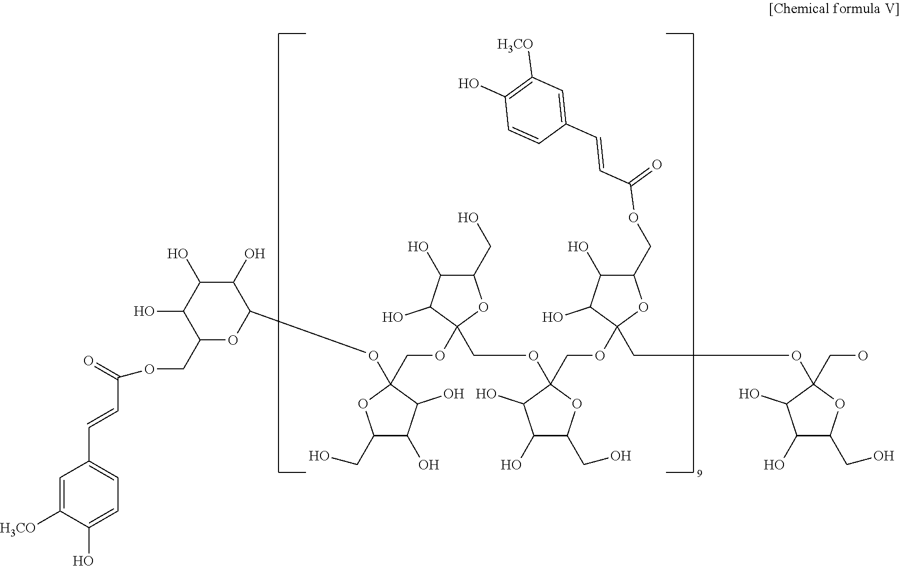

Further, the novel phytochemical-fructooligosaccharide conjugate according to the most preferred example of the present disclosure is composed of or contains a ferulic acid-fructooligosaccharide conjugate represented by a following chemical formula VI as a main component. The inventors of the present disclosure named the ferulic acid-fructooligosaccharide conjugate containing the compound represented by the following chemical formula VI as the main component as ‘FA-FOS II’. Based on the analysis of physical properties thereof, ‘FA-FOS II’ is soluble in water and hardly decomposes in oral, gastric and small intestine environments and most thereof reaches the large intestine. Further, the ferulic acid-fructooligosaccharide conjugate FA-FOS II is decomposed in a large intestine environment condition simulated by a human large intestine microbiota and released in the form of feruloyl glucose to be useful in preventing, ameliorating, or treating colorectal cancer.

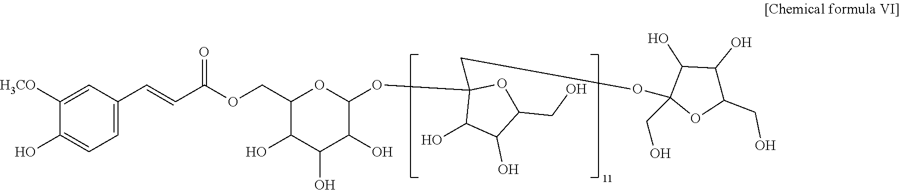

One aspect of the present disclosure relates to a pharmaceutically or food acceptable salt of the novel phytochemical-fructooligosaccharide conjugate described above. In the present disclosure, a pharmaceutically or food acceptable salt of the novel phytochemical-fructooligosaccharide conjugate may include an acid addition salt formed by free acid. The acid addition salt is produced by a conventional method, for example, by dissolving a compound in an excess acid aqueous solution, and precipitating the salt using a water-miscible organic solvent such as methanol, ethanol, acetone or acetonitrile. The same molar amount of the compound and an acid or alcohol (e.g., glycol monomethyl ether) in water may be heated and then the mixture may be evaporated to dryness, or the precipitated salt may be suctioned and filtered. In this connection, organic acids and inorganic acids may be used as free acids. Hydrochloric acid, phosphoric acid, sulfuric acid, nitric acid, tartaric acid, etc. may be used as inorganic acids. Methanesulfonic acid, p-toluenesulfonic acid, acetic acid, trifluoroacetic acid, citric acid, maleic acid, succinic acid, oxalic acid, benzoic acid, tartaric acid, fumaric acid, manderic acid, propionic acid, lactic acid, glycolic acid, gluconic acid, galacturonic acid, glutamic acid, glutaric acid, glucuronic acid, aspartic acid, ascorbic acid, carbonic acid, vanillic acid, hydroiodic acid, and the like may be used as the organic acids. Further, it is possible to produce a pharmaceutically or food acceptable metal salt using a base. The alkali metal or alkaline earth metal salt is obtained, for example, by dissolving a compound in an excess alkali metal hydroxide or alkaline earth metal hydroxide solution, filtering the undissolved compound salt, and evaporating and drying the filtrate. In this connection, sodium, potassium or calcium salts may be produced as a metal salt, and the corresponding silver salt is obtained by reacting an alkali metal or alkaline earth metal salt with a suitable silver salt (e.g., silver nitrate).

One aspect of the present disclosure relates to a method of producing a novel phytochemical-fructooligosaccharide conjugate. The novel phytochemical-fructooligosaccharide conjugate producing method according to an example of the present disclosure includes a step of (a) adding, dissolving, and heating a phytochemical in the form of phenolic acid and a catalyst for esterification reaction to a reaction solvent to perform an activation reaction of the phytochemical and obtaining a first reaction mixture containing a phytochemical in an activated form; and (b) adding fructooligosaccharide represented by a following general structural formula to the first reaction mixture, followed by heating in an inert gas atmosphere to perform an esterification reaction between fructooligosaccharide and phytochemical to obtain a second reaction mixture containing the phytochemical-fructooligosaccharide conjugate. Further, preferably, the method of producing the novel phytochemical-fructooligosaccharide conjugate according to an example of the present disclosure may further include (c) cooling and leaving the second reaction mixture to precipitate the phytochemical-fructooligosaccharide conjugate, followed by centrifugation and washing sequentially, thereby obtaining the purified phytochemical-fructooligosaccharide conjugate.

[General Structural Formula of Fructooligosaccharide]

Glu-(Fru)ₖ

In the general structural formula of fructooligosaccharide, ‘Glu’ denotes glucose, ‘Fru’ denotes fructose, and k denotes the number of fructoses linked to each other via a glycosidic bond, and is selected from an integer of 2 to 60, preferably selected from an integer of 5 to 49, and preferably selected from an integer of 9 to 41.

Structural features of the phytochemical-fructooligosaccharide conjugate finally obtained from the method of producing the novel phytochemical-fructooligosaccharide conjugate according to an example of the present disclosure refer to the above-described features, and detailed descriptions thereof will be omitted.

In the method for producing the novel phytochemical-fructooligosaccharide conjugate according to an example of the present disclosure, the phytochemical activation reaction in step (a) is preferably performed at 45 to 120° C. for 2 to 20 hr, and more preferably, at 50 to 80° C. for 5 to 18 hr. Further, as long as the reaction solvent used in step (a) is capable of dissolving a phytochemical in the form of phenolic acid, a catalyst for esterification reaction, and a fructooligosaccharide, the type thereof is not limited to the specific type. The reaction solvent may be selected from a variety of known organic solvents used in the field of organic synthesis. For example, the reaction solvent may be selected from polar organic solvents, and specific types thereof include dimethylsulfoxide, dimethylformamide, hexamethylphosphoramide, N-methylpyrrolidone, tetrahydrofuran, ethyl acetate, acetone, acetonitrile, and propylene carbonate. Further, the catalyst for the esterification reaction used in step (a) may be selected from various known catalysts used for esterification reaction between carboxylic acid and alcohol or esterification reaction between carboxylic acid and hydroxyl group. Specific types thereof include carbonyldiimidazole (N,N′-carbonyldiimidazole, CDI), dicyclohexylcarbodiimide, and 2-bromo-1-methylpyridinium iodide and the like.

Further, the molar ratio of phytochemical in the form of phenolic acid, the catalyst for esterification, and the fructooligosaccharide used in the producing method of the novel phytochemical-fructooligosaccharide conjugate according to an example of the present disclosure is preferably selected from the range of 1:1:1 to 40:40:1, and is preferably selected from the range of 2:2:1 to 35:35:1. For example, in the case of producing the ferulic acid-fructooligosaccharide conjugate FA-FOS I, the molar ratio of the ferulic acid, catalyst for esterification reaction, and fructooligosaccharide is preferably in a range of 20:20:1 to 35:35:1. Further, when producing the ferulic acid-fructooligosaccharide conjugate FA-FOS II, the molar ratio of the ferulic acid, catalyst for esterification reaction, and fructooligosaccharide is preferably in a range of 2:2:1 to 10:10:1.

Further, in the method for producing the novel phytochemical-fructooligosaccharide conjugate according to an example of the present disclosure, the esterification reaction of step (b) is preferably performed at 70 to 150° C. for 2 to 15 hr, and more preferably at 80 to 120° C. for 3 to 10 hr.

Further, in the method for producing a novel phytochemical-fructooligosaccharide conjugate according to an example of the present disclosure, a lower alcohol solvent having 2 to 5 carbon atoms may be used to wash the phytochemical-fructooligosaccharide conjugate in step (c). For example, examples of the lower alcohol solvent include ethanol, isopropyl alcohol, and the like.

In the method of producing novel phytochemical-fructooligosaccharide conjugate according to an example of the present disclosure, when the ferulic acid is used as a phytochemical in the form of phenolic acid, and carbonyldiimidazole (N,N-carbonyldiimidazole, CDI) is used as the catalyst for esterification reaction, the step (a) acts as an activation step of the ferulic acid and may be expressed as the following chemical reaction formula 1, and the step (b) may act as a step of grafting the activated ferulic acid to fructooligosaccharide, and may be expressed as a following chemical reaction formula 2.

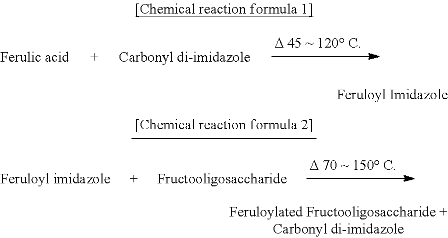

One aspect of the present disclosure relates to various pharmaceutical uses of the novel phytochemical-fructooligosaccharide conjugates, uses thereof for health functional foods, or uses thereof for cosmetics, based on the physiological activity thereof. An example of the present disclosure provides an anticancer composition containing a novel phytochemical-fructooligosaccharide conjugate or a pharmaceutically (or food) acceptable salt thereof as an active ingredient. Further, an example of the present disclosure provides a composition for prevention, amelioration, or treatment of colorectal cancer, the composition containing the novel phytochemical-fructooligosaccharide conjugate or a pharmaceutically (or food) acceptable salt thereof as an active ingredient. Further, an example of the present disclosure provides a composition for inhibiting metastasis of colorectal cancer, the composition containing the novel phytochemical-fructooligosaccharide conjugate or a pharmaceutically acceptable salt thereof as an active ingredient. Further, an example of the present disclosure provides an antioxidant composition containing the novel phytochemical-fructooligosaccharide conjugate or a pharmaceutically acceptable salt thereof as an active ingredient.

The novel phytochemical-fructooligosaccharide conjugate which is an active ingredient in the anticancer composition according to an example of the present disclosure is preferably FA-FOS I or FA-FOS II as described above. Further, the novel phytochemical-fructooligosaccharide conjugate, which is an active ingredient in the composition for colorectal cancer prevention, amelioration, or treatment according to an example of the present disclosure is preferably the aforementioned FA-FOS I or FA-FOS II, and is more preferably FA-FOS II. Further, the novel phytochemical-fructooligosaccharide conjugate which is an active ingredient in the composition for inhibiting metastasis of colorectal cancer according to an example of the present disclosure is preferably FA-FOS I or FA-FOS II, and is more preferably FA-FOS I. Further, the novel phytochemical-fructooligosaccharide conjugate which is an active ingredient in the antioxidant composition according to an example of the present disclosure is preferably FA-FOS I or FA-FOS II, and more preferably FA-FOS I.

The anticancer composition or the composition for colorectal cancer prevention, amelioration or treatment, the composition for inhibiting metastasis of colorectal cancer, or the antioxidant composition according to an example of the present disclosure may be embodied as a pharmaceutical composition, food additive, or food composition (especially, health functional food) or feed additives based on the use purpose and aspect thereof. Further, the antioxidant composition according to an example of the present disclosure may be embodied as a cosmetic composition for preventing the skin aging.

Further, a content of the novel phytochemical-fructooligosaccharide conjugate as an active ingredient in each of the various compositions according to an example of the present disclosure may be adjusted in various ranges according to the specific form, use purpose or aspect thereof.

The content of the novel phytochemical-fructooligosaccharide conjugate as an active ingredient in the pharmaceutical composition according to the present disclosure is not limited to a specific range, may be, for example, 0.01 to 99% by weight, preferably 0.5 to 50% by weight, more preferably, 1 to 30% by weight based on the total weight of the composition. Further, the pharmaceutical composition according to the present disclosure may further include additives such as a pharmaceutically acceptable carrier, excipient, or diluent in addition to the active ingredient. Carriers, excipients and diluents that may be contained in the pharmaceutical composition according to the present disclosure include lactose, dextrose, sucrose, sorbitol, mannitol, xylitol, erythritol, maltitol, starch, gum acacia, alginate, gelatin, calcium phosphate, calcium, silicate, cellulose, methyl cellulose, microcrystalline cellulose, polyvinyl pyrrolidone, water, methylhydroxybenzoate, propylhydroxybenzoate, talc, magnesium stearate and mineral oils. Further, the pharmaceutical composition according to the present disclosure may further contain at least one known active ingredient having anticancer activity, particularly, anti-colorectal cancer activity, in addition to the novel phytochemical-fructooligosaccharide conjugate. The pharmaceutical composition according to the present disclosure may be formulated into a formulation for oral administration or a formulation for parenteral administration by a conventional method. For formulation, diluents or excipients such as usually used fillers, extenders, binders, wetting agents, disintegrants, surfactants, etc. may be used. Solid preparations for oral administration include tablets, pills, powders, granules, capsules, and the like, and these solid preparations may be prepared by mixing at least one excipient such as starch, calcium carbonate, sucrose, lactose or gelatin with the active ingredient. Further, in addition to simple excipients, lubricants such as magnesium stearate and talc may be used. Liquid preparations for oral administration include suspensions, liquid solutions, emulsions, syrups, and the like. In addition to water and liquid paraffin, which are commonly used simple diluents, various excipients such as humectants, sweeteners, fragrances, and preservatives may be contained therein. Formulations for parenteral administration may include sterilized aqueous solutions, non-aqueous solutions, suspensions, emulsions, lyophilized formulations, and suppositories. As the non-aqueous solvent and the suspension solvent, propylene glycol, polyethylene glycol, vegetable oil such as olive oil, and injectable ester such as ethyl oleate may be used. As a base for suppositories, witepsol, macrogol, tween 61, cacao butter, laurinum, glycerogelatin, and the like may be used. Further, the composition may be preferably formulated according to each disease or component using an appropriate method in the art or using a method disclosed in Remington's Pharmaceutical Science (latest edition), Mack Publishing Company, Easton Pa. The pharmaceutical composition according to the present disclosure may be administered orally or parenterally to mammals, including humans, depending on the intended method. The parenteral administration methods include skin external use, intraperitoneal injection, rectal injection, subcutaneous injection, intravenous injection, intramuscular injection or intrathoracic injection. The dosage of the pharmaceutical composition according to the present disclosure is not limited to the specific amount as long as the dosage is a pharmaceutically effective amount. The range thereof may vary depending on the patient's weight, age, sex, health status, diet, administration time, administration method, excretion rate, and disease severity. The usual daily dosage of the pharmaceutical composition according to the present disclosure is not limited to the specific amount, but is preferably 10 to 9000 mg/kg, more preferably 100 to 5000 mg/kg based on the active ingredient. The composition may be administered once or in several divided manners per one day.

Further, the content of the novel phytochemical-fructooligosaccharide conjugate as an active ingredient in the food composition according to the present disclosure is 0.01 to 99% by weight, preferably 0.1 to 50% by weight, more preferably 0.5 to 25% by weight based on the total weight of the composition but is not limited thereto. The food composition according to the present disclosure includes the form of pills, powders, granules, infusions, tablets, capsules, or liquids. Examples of specific foods include meat, sausage, bread, chocolate, candy, snacks, confectionery, pizza, and ramen, other noodles, gums, dairy products including ice cream, various soups, beverages, tea, functional water, drinks, alcoholic beverages and vitamin complexes. All health functional foods in the usual sense are included therein. The food composition according to the present disclosure may contain a food acceptable carrier, various flavoring agents, or natural carbohydrates as additional ingredients in addition to the active ingredient. Further, the food composition according to the present disclosure may contain a variety of nutrients, vitamins, electrolytes, flavoring agents, colorants, pectic acid and its salts, alginic acid and its salts, organic acids, protective colloidal thickeners, pH regulators, stabilizers, preservatives, glycerin, alcohol, carbonation agents used in carbonated beverages, and the like.

Further, the cosmetic composition according to the present disclosure may be produced in any formulation commonly produced in the art, For example, the cosmetic composition according to the present disclosure may be formulated into a solution, suspension, emulsion, paste, gel, cream, lotion, powder, soap, surfactant-containing cleansing, oil, powder foundation, emulsion foundation, wax foundation and spray, etc. but is not limited thereto. Specifically, the cosmetic composition according to the present disclosure may be produced in the form of a flexible lotion, nutritional lotion, nutritional cream, massage cream, essence, eye cream, cleansing cream, cleansing foam, cleansing water, pack, spray or powder.

Hereinafter, the present disclosure will be described in more detail based on examples. However, the following examples are only intended to clearly illustrate the technical characteristics of the present disclosure, and do not limit the scope of protection of the present disclosure.

1. Producing and Analysis of Substitution Degree of Ferulic Acid-Fructooligosaccharide Conjugate

(1) FA-FOS I Producing

Dimethyl sulfoxide (DMSO) and ferulic acid were added to the reaction vessel, and ferulic acid was dissolved in dimethyl sulfoxide (DMSO) to produce a 5% (w/v) ferulic acid solution. Thereafter, carbonyldiimidazole (N,N′-carbonyldiimidazole, CDI) was added to the reaction vessel and the mixture was stirred at about 60° C. for about 16 hours to activate ferulic acid. The activated form of ferulic acid is feruloyl imidazole. Thereafter, fructooligosaccharide [a polymer in which about 2 to 60 D-fructose residues are connected to each other via β (2→1) bonds to the reaction vessel, and one D-glucose at one end thereof is connected, via α (1→2) bond, to D-fructose) was added thereto and dissolved, and the reaction vessel was sealed. The molar ratio of ferulic acid, carbonyldiimidazole and fructooligosaccharide present in the sealed reaction vessel was 32:32:1. Thereafter, the temperature of the reaction vessel was increased to 90° C., and the mixture was continuously stirred for about 7 hours in an inert gas (nitrogen) atmosphere to perform a reaction to graft the activated ferulic acid to the fructooligosaccharide. After the reaction was completed, the reaction mixture was cooled to room temperature, and ice-cold iso-propyl alcohol was added thereto in an amount of about 3 times of the volume of the reaction mixture, and the mixture was allowed to stand at about 4° C. overnight to precipitate the ferulic acid-fructooligosaccharide conjugate (FA-FOS). Thereafter, the reaction mixture was centrifuged (4000×g, 20 minutes) to obtain a pellet, and the obtained pellet was washed with iso-propyl alcohol about 3 times to remove unreacted ferulic acid, carbonyldiimidazole and feruloyl imidazole and then was washed again with distilled water to obtain a final reaction product, that is, the ferulic acid-fructooligosaccharide conjugate I (abbreviated as ‘FA-FOS I’). The final reaction product FA-FOS I had a producing yield of about 89%.

(2) FA-FOS II Producing

Dimethyl sulfoxide (DMSO) and ferulic acid were added to the reaction vessel, and ferulic acid was dissolved in dimethyl sulfoxide (DMSO) to produce a 5% (w/v) ferulic acid solution. Thereafter, carbonyldiimidazole (N,N′-carbonyldiimidazole, CDI) was added to the reaction vessel and the mixture was stirred at about 60° C. for about 16 hours to activate ferulic acid. The activated form of ferulic acid is feruloyl imidazole. Thereafter, fructooligosaccharide [a polymer in which about 2 to 60 D-fructose residues are connected to each other via β (2→1) bonds to the reaction vessel, and one D-glucose at one end thereof is connected, via α (1→2) bond, to D-fructose) was added thereto and dissolved, and the reaction vessel was sealed. The molar ratio of ferulic acid, carbonyldiimidazole and fructooligosaccharide present in the sealed reaction vessel was 4:4:1. Thereafter, the temperature of the reaction vessel was increased to 90° C., and the mixture was continuously stirred for about 7 hours in an inert gas (nitrogen) atmosphere to perform a reaction to graft the activated ferulic acid to the fructooligosaccharide. After the reaction was completed, the reaction mixture was cooled to room temperature, and ice-cold iso-propyl alcohol was added thereto in an amount of about 3 times of the volume of the reaction mixture, and the mixture was allowed to stand at about 4° C. overnight to precipitate the ferulic acid-fructooligosaccharide conjugate (FA-FOS). Thereafter, the reaction mixture was centrifuged (4000×g, 20 minutes) to obtain a pellet, and the obtained pellet was washed with iso-propyl alcohol about 3 times to remove unreacted ferulic acid, carbonyldiimidazole and feruloyl imidazole to obtain a final reaction product, that is, the ferulic acid-fructooligosaccharide conjugate II (abbreviated as ‘FA-FOS II’). The final reaction product FA-FOS II had a producing yield of about 89%.

(3) Analysis of Ferulic Acid Substitution Degree of FA-FOS I and FA-FOS II

The degree of substitution of ferulic acid in FA-FOS I and FA-FOS II is defined as the average number of attached substituents ferulic acid per a basic unit of fructooligosaccharide. Each of FA-FOS I and FA-FOS II were added to 2M NaOH aqueous solution, and cultured for 2 hr to release a ferulate group from the conjugate. Thereafter, a 6N aqueous HCl solution was added to the NaOH aqueous solution to adjust the pH to 2, and the released ferulate group was acidified into the form of free ferulic acid. Thereafter, ferulic acid was extracted three times with ethyl acetate, and the amount of the released ferulic acid was quantified using HPLC, and the degree of substitution of ferulic acid was calculated using a following equation.

$$The degree of substitution ( DS ) = \frac{162 \times ( \% Ferulate )}{( 100 \times 194.19 ) - ( ( 194.19 - 1 ) \times ( \% Ferulate ) )}$$

162: molecular weight of the basic unit of fructooligosaccharide

194.19: Molecular weight of ferulic acid

% Ferulate: Weight % of ferulate based on the total weight of the conjugate

Table 1 below shows % Ferulate and the degree of substitution of ferulic acid of FA-FOS I and FA-FOS II.

TABLE 1

| Samples | % Ferulate | Degree of substitution |
|---|---|---|
| FA-FOS I | 20.93 | 0.2206 |
| FA-FOS II | 2.575 | 0.02 |

As shown in the above table, the substitution degree of the ferulic acid of FA-FOS I was approximately 10 times higher than that of FA-FOS II. FA-FOS I was not soluble in water and organic solvents, whereas FA-FOS II was immediately soluble in water and organic solvents. It was identified that the higher the degree of substitution of ferulic acid, the lower the solubility of FA-FOS in water. FA-FOS I was insoluble in water due to its amphiphilicity, and was immediately dissolved in ionic liquids such as tetrabutyl phosphonium acetate and tetra butyl ammonium acetate.

2. Structural Characterization of Ferulic Acid-Fructooligosaccharide Conjugate

(1) FA-FOS I

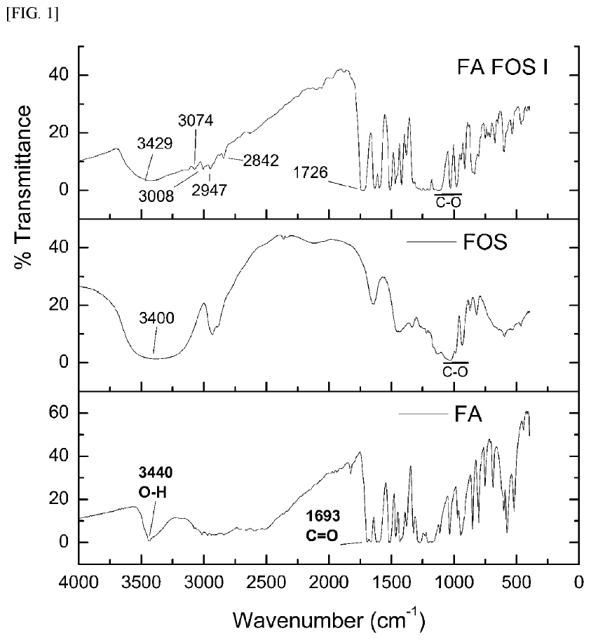

FIG. 1 shows the spectral results of ferulic acid (FA), fructooligosaccharide (FOS) and FA-FOS I analyzed by FTIR spectroscopy (Fourier-transform infrared spectroscopy). In FIG. 1, FA-FOS I is marked as ‘FA FOS I’. As shown in FIG. 1, in FA-FOS I, the intensity of the gentle peak at 3400 cm⁻¹ of fructooligosaccharide (FOS) significantly decreased as the number of hydroxyl groups decreased. The peak at 1726 cm⁻¹ of FA-FOS I represents a substitution or esterification reaction, and the two peaks occurring at 2947 cm⁻¹, 3008 cm⁻¹, and 2842 cm⁻¹ are due to methyl and methylene C—H stretching associated with feruloyl substitution. The peak between 900 and 1200 cm⁻¹ of FOS is due to C—O stretching, and the peak at 3074 cm⁻¹ of FA-FOS I is due to C—H stretching related to methane hydrogen of the ring.

Further, ferulic acid (FA), fructooligosaccharide (FOS), and FA-FOS I were analyzed with a solid state nuclear magnetic resonance (NMR) spectroscopy under a 13C (L armor frequency: 400.25 MHz) condition. Since FA-FOS I is not soluble in organic solvents such as dimethyl sulfoxide (DMSO), solid state nuclear magnetic resonance (NMR) was performed to explain its structure. In the 13C solid state nuclear magnetic resonance (NMR) spectrum of FA-FOS I, the aromatic carbon of ferulic acid was observed between 100 ppm and 150 ppm, and the carbonyl carbon (—COOH) transfer of ferulic acid was observed in the region exceeding 150 ppm. The peak shift of pure ferulic acid was observed at 173.13 ppm, but in the case of FA-FOS I, the peak shift was observed at 164.17 ppm due to the formation of ester bonds between fructooligosaccharide (FOS) and ferulic acid. Further, in pure fructooligosaccharide (FOS), the aliphatic carbon was observed based on a number of narrow peaks over 50 ppm to 100 ppm, but in the case of FA-FOS I, the aliphatic carbon of fructooligosaccharide was observed based on a single broad peak at 55.17 ppm. This result is because in FA-FOS I, the terminal glucose of fructooligosaccharide (FOS) and ferulic acid are connected to each other, and four fructoses of fructooligosaccharide (FOS) are bound to ferulic acid. The multiplets observed in the 13C solid state NMR spectrum of FA-FOS I are as follows. 13C NMR (101 MHz): d=263.9, 251.1, 233.4, 223.3, 217.4, 180.4, 164.4, 151.9, 148.1, 144.7, 142.2, 133.6, 124.2, 117.6, 109.0, 76.7, 65.0, 55.3, 43.1, 34.1, 24.4, 18.5 ppm

Further, ferulic acid (FA), fructooligosaccharide (FOS), and FA-FOS I were analyzed using solid state nuclear magnetic resonance (NMR) spectroscopy under 1H (L armor frequency: 400.66 MHz, spin rate: 14 KHZ) condition. In the 1H solid state nuclear magnetic resonance (NMR) spectrum of FA, the peak of 13.19 ppm corresponds to the chemical shift of carboxylic acid (R—COOH) present in ferulic acid. On the other hand, in FA-FOS I, ferulic acid binds to glucose and fructose as sugar molecules of fructooligosaccharide (FOS), such that the 13.19 ppm peak did not exist and a strong peak was observed at 4.94 ppm. The strong peak at 4.94 ppm indicates the —OH group bonded to the aromatic group of ferulic acid in FA-FOS I. Further, the peak at 6.94 ppm observed in FA-FOS I corresponds to —H bonded to the aromatic ring of ferulic acid, and does not exist in the NMR spectrum of pure fructooligosaccharide. Further, the apparent peak of 2.56 ppm observed in FA-FOS I is due to the carbonyl group present when ferulic acid is coupled to fructooligosaccharide in a substituting manner. This is strong evidence for the conjugation between ferulic acid and fructooligosaccharide (FOS).

Further, ferulic acid (FA), fructooligosaccharide (FOS) and FA-FOS I were analyzed by XRD (X-ray Diffraction) spectroscopy. As a result, FA FOS I showed strong diffraction peaks at about 17.650 (2θ) and 25.55° (2θ), and showed small and broad peaks at 10.5°, 12.70°, 20.31°, 23.73°, and 28.07° (2θ). FA FOS I appeared as a mixture of the A-type crystalline phase and the amorphous phase, and had more crystal structure than the FOS had when the peaks thereof were compared to the peaks of FOS. From the above results, it may be seen that the linear fructose present in FA FOS I contributes to the amorphous region, and conjugation of FOS with ferulic acid at the terminal glucose thereof and conjugation of FOS with fructose at the fourth fructose thereof contribute to the crystal region.

Further, FA-FOS I was analyzed with a UV-Vis spectroscopy (Ultraviolet-visible spectroscopy) and a fluorescence spectrophotometer. As a result, FA-FOS I absorbed light at 280 nm and emitted light at 310 nm.

Further, thermogravimetric analysis (TGA) on ferulic acid (FA), fructooligosaccharide (FOS) and FA-FOS I was performed. As a result, FOS showed a weight loss of about 41.06% at 238° C., and FA-FOS I showed a weight loss of about 41.5% at 400° C. It may be seen that FA-FOS I has higher thermal stability compared to FOS.

Further, the zeta potential of FA-FOS I was measured, and the particle size thereof was measured using Dynamic Light Scattering (DLS). As a result, the zeta potential of FA-FOS I was found to be −14.5±4.88 mV, and the average particle diameter thereof was 1.839±0.339 μm. The low zeta potential of FA-FOS I seems to be caused by grafting of ferulic acid, and FA-FOS I is found to self-aggregate into a higher-order structure that creates a homogeneous suspension in water. Further, the polydispersity index of FA-FOS I measured via DLS is 0.243, indicating homogeneity.

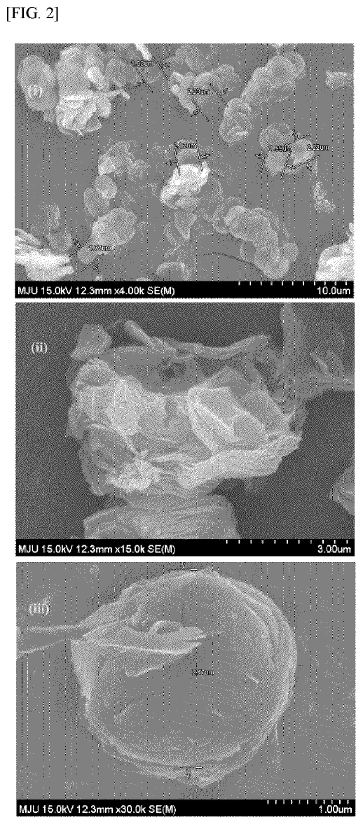

FIG. 2 is an image result of FA-FOS I analyzed by SEM (Scanning Electron Microscopy). In FIG. 2, i is a low-magnification image showing the disk-shaped structure, ii is a cross-sectional image of disk-shaped particles, and iii is a high-magnification image showing disk-shaped fine particles. As shown in FIG. 2, FA-FOS I has a disk-shaped structure with an average diameter of about 2 μm due to self-aggregation. Further, according to iii of FIG. 2, FA-FOS I which has a disk-shaped structure is formed by a layered sheet-like assembly with a cavity.

From the results of analyzing the structural characteristics of FA-FOS I, it may be seen that the main component of FA-FOS I is a ferulic acid-fructooligosaccharide conjugate represented by a following chemical formula V.

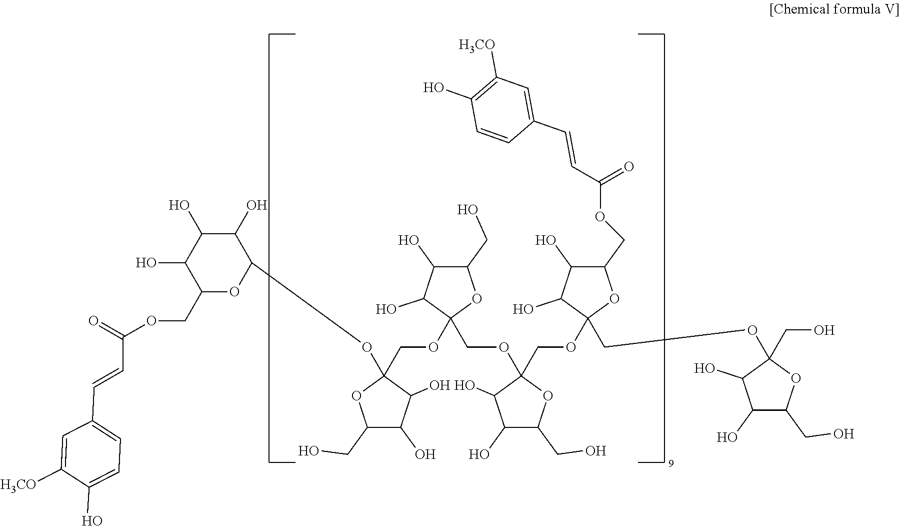

(2) FA-FOS II

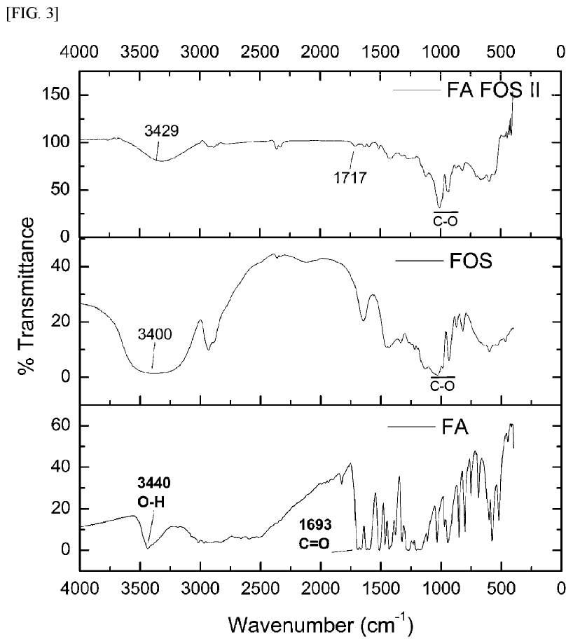

FIG. 3 shows the spectral results of ferulic acid (FA), fructooligosaccharide (FOS) and FA-FOS II analyzed by FTIR spectroscopy (Fourier-transform infrared spectroscopy). In FIG. 3, FA-FOS II is indicated as ‘FA FOS II’. As shown in FIG. 3, in FA-FOS II, the intensity of the gentle peak at 3400 cm⁻¹ of fructooligosaccharide (FOS) decreased as the number of hydroxyl groups decreased. The small peak at 1717 cm⁻¹ of FA-FOS II represents an esterified carbonyl group, and the peak between 900 and 1200 cm⁻¹ is due to C—O stretching derived from the FOS molecule.

Further, FA-FOS II was analyzed with a nuclear magnetic resonance (NMR) spectroscopy under 13C condition and a nuclear magnetic resonance (NMR) spectroscopy under 1H condition. The multiplets observed in the 13C nuclear magnetic resonance (NMR) spectrum of FA-FOS II are as follows. 13C NMR (DMSO-d6) δ: 104.25, 103.52, 103.38, 81.89, 76.88, 74.46, 62.33, 61.79, 40.64, 25.76. The multiplets observed in the 1H nuclear magnetic resonance (NMR) spectrum of FA-FOS II are as follows. 1H NMR (DMSO-d6) δ: 6.82-6.61 (m, OH), 5.11 (d, J=5.7 Hz, 11H), 4.67 (d, J=6.5 Hz, 10H), 4.56 (t, J=5.3 Hz, 11H), 4.01 (t, J=7.3 Hz, 10H), 3.82-3.72 (m, 12H), 3.59 (d, J=8.5 Hz, 11H), 3.55 (s, 6H), 3.43 (d, J=19.1 Hz, 15H), 3.35 (s, 11H), 2.47 (d, J=1.8 Hz, 0H), 2.46 (s, 1H), 2.45 (d, J=1.9 Hz, 0H), 1.00 (s, 1H), 0.98 (s, 1H).

From the results of analyzing the structural characteristics of FA-FOS II, it may be seen that the main component of FA-FOS II is a ferulic acid-fructooligosaccharide conjugate represented by a following chemical formula VI. Further, based on a result of analyzing FA-FOS II by LC-MS (Liquid Chromatography-Mass Spectrometry) and MS-MS (Mass spectroscopy-mass spectroscopy), the number of fructoses present in the main chain of FA-FOS II was 3 to 24 and was variously distributed.

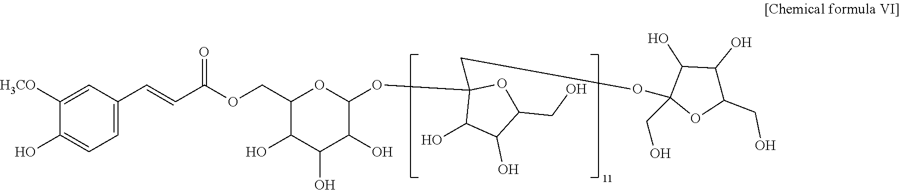

3. Stability and Decomposition Characteristics of Ferulic Acid-Fructooligosaccharide Conjugate in Oral-Gastrointestinal Tract Simulation Conditions, Etc.

(1) Stability Test in Oral-Gastrointestinal Tract Simulation Conditions

The stability characteristics were measured by HPLC when each of FA-FOS I and FA-FOS II was added to each of simulated salivary fluid (SS), simulated gastric fluid (SGF) and simulated small intestine fluid (SSI) at 37° C. and incubated therein for 0 minutes, 15 minutes, 30 minutes, and 1 hr to 4 hr. Compositions of simulated salivary fluid (SS), simulated gastric fluid (SGF), and simulated small intestine fluid (SSI) used in the experiments to measure the stability characteristics of the ferulic acid-fructooligosaccharide conjugate under simulation conditions of the oral cavity-gastrointestinal tract are as follows.

<Simulated Salivary Fluid Composition>

Based on 100 ml of the simulated salivary fluid, the simulated salivary fluid contains CaCl₂.2H₂O 0.0228%, NaCl 0.1017%, Na₂HPO₄ 0.0204%, MgCl₂ 0.0061%, K₂CO₃ 0.0603%, Na₂HPO₄ 0.0273%, α-amylase 200 units, and Lysozyme 0.0015% and has pH 7.4

<Simulated Gastric Fluid Composition>

Based on 100 ml of the simulated gastric fluid, the simulated gastric fluid contains sodium taurocholate 80 μM, lecithin 20 μM, pepsin 0.01%, lysozyme 0.01%, and sodium chloride 34.2 mM, and has pH 1.6

<Simulated Small Intestine Fluid Composition>

Based on 100 ml of the simulated small intestine fluid, the simulated small intestine fluid contains sodium taurocholate 3 mM, lecithin 0.2 mM, maleic acid 19.12 mM, sodium hydroxide 34.8 mM, sodium chloride 68.62 mM, pancreatin (4 USP/mg) 0.05 g, and has pH 6.5

(2) Digestion Characteristics Experiment in Large Intestine Environment Conditions Simulated by Human Microbiota

0.05 g of each of sterilized FA-FOS I, FA-FOS II and fructooligosaccharide (FOS) was added to each vial containing 10 ml of sterilized anaerobic minimal basal medium. Under anaerobic conditions, human feces were inoculated thereto at the amount of 1% (w/v) and the vial was sealed. Thereafter, the sealed vial was incubated at 37° C. for 12 hr, 24 hr, and 36 hr to 48 hr and the mixture was stirred intermittently. After the culture was completed, the cultured medium was centrifuged to collect the supernatant, and a 6N aqueous HCl solution was added to the collected supernatant to adjust the pH to 2, and free ferulic acid was extracted 3 times with 2 times of the volume of ethyl acetate. Thereafter, the extract was evaporated under a nitrogen atmosphere to remove ethyl acetate and was dissolved in 50% methanol aqueous solution to prepare a sample for analysis. Then, the analysis sample was analyzed via HPLC to determine the amount of free ferulic acid released under the simulated large intestine environment conditions. The composition of the anaerobic minimal basal medium used in the experiment is as follows.

<Composition of Anaerobic Minimal Basal Medium>

Based on 100 ml of the anaerobic minimal basal medium, the anaerobic minimal basal medium contains Peptone 0.2 g, Yeast extract 0.1 g, NaCl 0.01 g, K₂HPO₄ 0.004 g, KH₂PO₄ 0.004 g, MgSO₄.7H₂O 0.001 g, CaCl₂.2H₂O 0.001 g, NaHCO₃ 0.2 g, Bile salts 0.05 g, L-Cysteine HCl 0.05 g, Tween 800.2 ml, 0.05% Resazurin solution A 0.1 ml, Hemin 0.0005 g, 0.02 Mm Vitamin K₁ 10 μl

(3) Stability Test Against Digestive Enzymes in the Intestine

Each of 250 mg of FA-FOS I and FA-FOS II were added to each of the intestinal digestive enzyme solution and incubated therein at 37° C. for 12 hr, 24 hr, 36 hr, and 48 hr. The intestinal digestive enzyme solution is a liquid mixed enzyme solution as produced by adding Cellulase, Endo-galactouranase, endo-carbohydrase, exo-glycosidase and Feruloyl esterase produced by bacteria inhabiting in the large intestine to 0.02% (w/v) sodium azide solution. and has a pH of 6. Specifically, in the intestinal digestive enzyme solution, Driselase (Amano enzymes, Japan) 50 mg, Protease M Amano (Amano enzymes, Japan) 50 mg, DEPOL 670 L (Biocatalyst, UK) 50 μl, DEPOL 740 L (Biocatalyst, UK) 50 μl and 0.02% (w/v) sodium azide were dissolved in 5 ml of MOPS buffer at pH 6.0. Thereafter, the culture solution was centrifuged to collect the supernatant. The pH thereof was adjusted to 2 by adding a 6N aqueous HCl solution to the collected supernatant. The free ferulic acid was extracted therefrom 3 times with 3 times of the volume of ethyl acetate. Thereafter, the extract was evaporated under a nitrogen atmosphere to remove ethyl acetate and was dissolved in 50% methanol aqueous solution to prepare a sample for analysis. Then, the analysis sample was analyzed using HPLC.

(4) Experiment Result

Table 2 below shows the results of stability tests in the oral cavity-gastrointestinal tract simulation conditions, digestion characteristics tests in large intestine environment conditions simulated by human microbiota, and stability tests against the intestinal digestive enzymes for FA-FOS I. Further, the following Table 3 shows the results of stability tests in the oral cavity-gastrointestinal tract simulation conditions, digestion characteristics tests in large intestine environment conditions simulated by human microbiota, and stability tests against the intestinal digestive enzymes for FA-FOS II.

TABLE 2

| simulation condition | stability*(%) | half-life**(hr) |
|---|---|---|
| simulated salivary fluid | 99.06 | >4 |
| simulated gastric fluid | 100 | >4 |
| simulated small intestine | 99.08 | >4 |
| fluid | | |
| simulated large intestine | 100 | >48 |
| environment | | |
| intestinal digestive enzyme | 99.17 | >24 |
| fluid | | |

TABLE 3

| simulation condition | stability*(%) | half-life**(hr) |
|---|---|---|
| simulated salivary fluid | 100 | >4 |
| simulated gastric fluid | 100 | >4 |
| simulated small intestine | 99.96 | >4 |
| fluid | | |
| simulated large intestine | 100 (Digested and | <24 |
| environment | metabolized) | |
| intestinal digestive enzyme | 54.39 | 36 |
| fluid | | |

*Stability was expressed as a percentage between the amount of ferulic acid contained in the ferulic acid-fructooligosaccharide conjugate before cultivation and the amount of free ferulic acid released from the ferulic acid-fructooligosaccharide conjugate after cultivation at 37° C.

**Half-life was expressed as the time duration for which half of the ferulic acid contained in the ferulic acid-fructooligosaccharide conjugate was released into the medium.

According to the stability and half-life results of FA-FOS I and FA-FOS II in the oral cavity-gastrointestinal tract simulation conditions as shown in Tables 2 and 3, FA-FOS I and FA-FOS II were hardly degraded in simulated salivary fluid (SS), simulated gastric fluid (SGF) and simulated small intestine fluid (SSI).

As shown in Table 2, FA-FOS I was found to be hardly degraded by human large intestine microbiota.

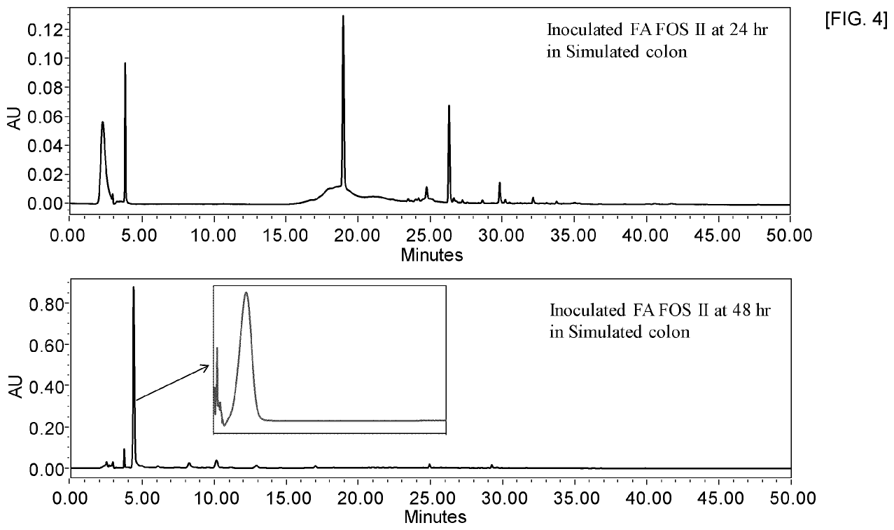

FIG. 4 shows the results of HPLC analysis of the decomposition behavior when FA-FOS II is cultured in the large intestine environment condition simulated by human large intestine microbiota. As shown in Table 3 and FIG. 4, FA-FOS II was decomposed into the ferulate form by the human large intestine microbiota and was released. Further, the final metabolites obtained when FA-FOS II was cultured in a large intestine environment condition simulated by a human large intestine microbiota was analyzed by LC-MS (Liquid Chromatography-Mass Spectrometry) and MS-MS (Mass spectroscopy-mass spectroscopy). Thus, the final metabolite of FA-FOS II in the human large intestine environment was identified as feruloyl glucose.

As shown in Tables 2 and 3, FA-FOS I was more resistant to digestive enzymes in the intestine than FA-FOS II was, and the amount of free ferulic acid released from FA-FOS I was very small.

4. Anticancer Activity of Ferulic Acid-Fructooligosaccharide Conjugate in In-Vitro Experiment

(1) Observation and Analysis of Cancer Cell Death-Inducing Progress

Normal human large intestine cell line such as CCD 18Co (Normal Human colon fibroblast primary cell line), colorectal cancer cell line such as HT-29 (Colorectal adenocarcinoma, capable of inducing tumor in mice), or metastasis colorectal cancer cell lines such as Lovo (Duke's type C, grade IV, Colorectal adenocarcinoma capable of metastasis) were treated with oxaliplatin, 5-fluorouracil, FA-FOS I, FA-FOS II and ferulic Acid (FA) and then cell death-inducing progress was observed. Specifically, the cell lines cultured in each well of a 96-well plate were dispensed at a density of 18,000 cells per well and cultured overnight, and then the cells were treated with drugs dissolved in various concentrations in the culture medium, and were incubated for 72 hr under a temperature of 37° C. and 5% CO₂ condition in a humidified incubator. After completion of the culture, the medium was removed and replaced with 100 μl of PBS, and 10 of Cell Counting Kit-8 (CCK-8; Dojindo, Japan) solution was added thereto, and the cells were incubated for 3 hr for reaction. Thereafter, the degree of color development of the reaction solution was measured based on absorbance at 450 nm, and the number of cell proliferation was calculated according to the instructions of the producing company.

HT-29 cells were cultured in McCoy's 5A medium, LoVo cells were cultured in Ham's F-12K (Kaigh's) medium, CCD 18Co cells were cultured in DMEM. Before use, 10% Fetal bovine serum and 100 U/ml penicillin, streptomycin were added to all media.

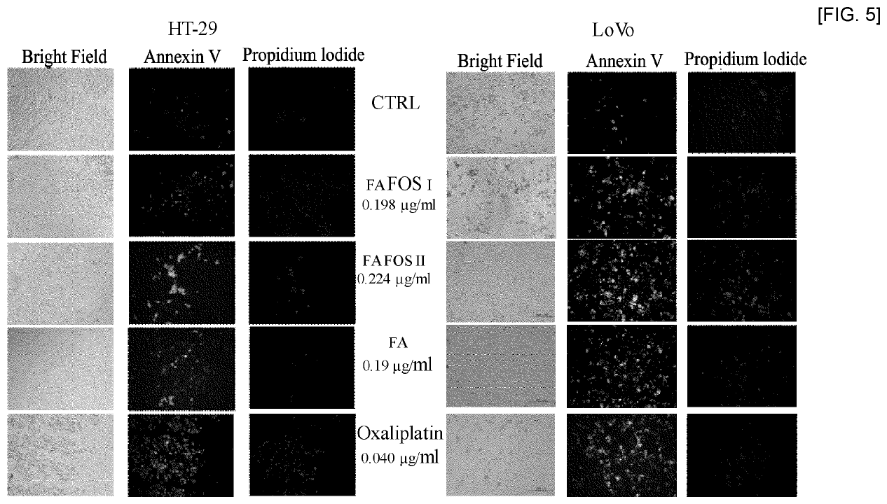

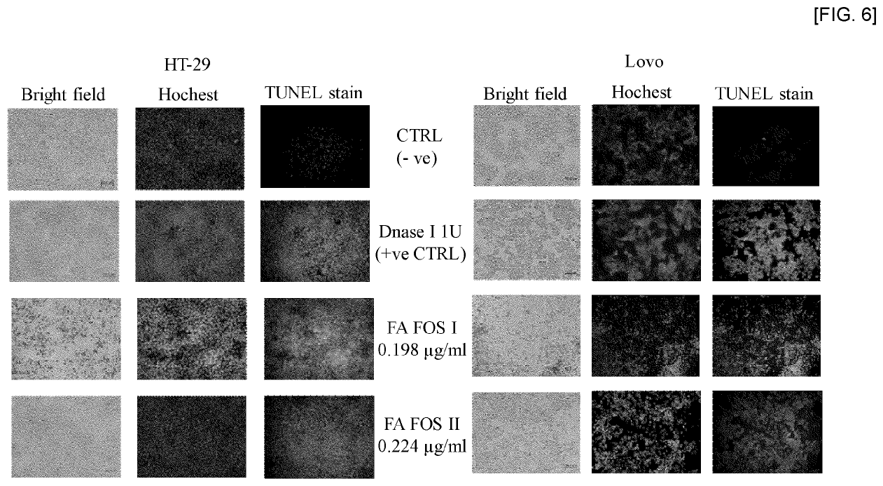

FIG. 5 shows the result of observation of cancer cell death-inducing progress using a fluorescence microscope after staining with Annexin V and Propidium iodide when a HT-29 cell line and Lovo cell line are treated with oxaliplatin, FA-FOS I, FA-FOS II and ferulic acid (FA). Further, FIG. 6 shows the result of observation of cancer cell death-inducing progress using a fluorescence microscope after TUNEL assay (terminal deoxynucleotidyl transferase-dUTP nick end labeling) and additional Hoechst 33342 staining when HT-29 cell line and Lovo cell line are treated with FA-FOS I and FA-FOS II. Further, the following Table 4 shows the IC₅₀ (half maximal inhibitory concentration) values and the Selectivity Index (SI) values of the treated drugs for various cell lines.

TABLE 4

| | IC₅₀ (Unit: μg/ml) and SI | | | | |
|---|---|---|---|---|---|
| Cell Line | Oxaliplatin | 5-Fluorouracil | FA-FOS I | FA-FOS II | FA |
| CCD 18Co | 0.100 | 0.014 | 5.926 | 6.723 | 0.583 |
| HT-29 | 0.015 | 0.008 | 0.127 | 3.942 | 0.200 |
| | (SI: 6.631) | (SI: 1.685) | (SI: >46.490) | (SI: >1.706) | (SI: >2.918) |
| Lovo | 0.068 | 0.074 | 0.111 | 3.054 | 0.244 |
| | (SI: 1.473) | (SI: 0.186) | (SI: >53.571) | (SI: >2.201) | (SI: >2.389) |

* SI = Selectivity Index, IC₅₀ for Normal cell (CCD 18Co)/IC₅₀ for Cancer cell

As shown in FIG. 5, FIG. 6 and Table 4 above, FA-FOS I and FA-FOS II showed excellent colorectal cancer cell death effects. In particular, FA-FOS I was identified as having a higher selectivity index when compared to the commercial drugs oxaliplatin and 5-fluorouracil. Further, FA-FOS I showed a lower IC₅₀ value than FA showed.

(2) Analysis of Cell Death-Inducing Mechanism of Ferulic Acid-Fructooligosaccharide Conjugate FA-FOS I

To understand the mechanism of cell death-inducing by FA-FOS I, the LoVo cell line was treated with FA-FOS I at a concentration of 0.198 μg/ml for 12 hr, and then gene expression was profiled. Total m-RNA was extracted from the treated cells using a Qiagen m-RNA extraction and purification kit, and analyzed using a Qiagen RT2 Profiler PCR array Human apoptosis kit. Table 5 below shows the profiling results of genes expressed in relation to cell death in LoVo cell lines treated with FA-FOS I.

TABLE 5

| | Fold Regulation | | Fold Regulation |
|---|---|---|---|
| | (compared to | | (compared to |
| | FA-FOS I | | FA-FOS I |
| gene | non-treatment) | gene | non-treatment) |
| CASP 1 | 1.56 | TNFSF8 | 1.49 |
| CD27 | 1.58 | PYCARD | 1.11 |
| CD40 | 4.38 | BCL2A1 | −33.55 |
| CD40LG | 2.97 | BCL2L10 | −2.49 |
| CIDEA | 1.49 | BCL2 | −1.50 |
| FASLG | 1.79 | CASP5 | −1.50 |
| IL10 | 3.79 | CRADD | −1.69 |
| LTA | 2.62 | TNFRSF25 | −1.81 |
| TNFRSF11B | 1.95 | TNF | −1.16 |
| TNFRSF9 | 32.94 | | |

As shown in Table 5 above, when the LoVo cell line was treated with FA-FOS I, 12 genes were up-regulated and 7 genes were down-regulated. TNFRSF9, the upregulated gene in Table 5 is called CD137 belonging to the TNF-receptor superfamily, and is involved in clonal expansion, survival and development of T cells (immune cells), and activates T cells to induce an immune response, induces the secretion of IL-2 (Inter Lukin 2) and invasion of immune cells, and removes tumors. Upregulation of TNFRSF9 provides clues to the possibility of FA-FOS I to eliminate tumor/cancer cells in vivo through immune cell mediation.

Cell Cycle Arrest examines the proliferation of tumor cells by controlling a cell cycle checkpoint regulator called cyclin and is one of the functions of anticancer drugs. To investigate the function of FA-FOS I in cell cycle arrest, each of the LoVo cell line and the HT-29 cell line was seeded on a culture plate and starved for 48 hr using a culture medium without FBS for synchronization, and was treated with FA-FOS I and FA at different concentrations. After culturing the LoVo cell line for 24 hr and culturing the HT-29 cell line for 72 hr, the proliferated cells were stained with DAPI, and the number of cells was analyzed by FACS. FA-FOS I dose-dependently arrested the cell cycle in the G0 phase of the cell cycle. Specifically, when FA-FOS I was applied, at a concentration of 0.198 μg/ml, to HT-29 cell line, about 91.22% was arrested on G0 phase. When FA-FOS I was applied, at a concentration of 0.988 μg/ml, to HT-29 cell line, about 94.16% was arrested on the G0 phase. Further, when FA-FOS I was applied, at a concentration of 0.198 μg/ml, to LoVo cell line, about 62.74% was arrested on G0 phase. When FA-FOS I was applied, at a concentration of 0.988 μg/ml, to LoVo cell line, about 79.52% was arrested on G0 phase. This result is very high compared to 48.64% of the control (no treatment).

Further, to verify the function of FA-FOS I on cell cycle arrest, a total protein was extracted from LoVo cell lines, and expression profiling of cyclin B1, E and cyclin-dependent kinase inhibitory protein p21 was analyzed by densitometry. As a result, FA-FOS I up-regulates the expression of p21 which inhibits all cyclins that regulate the cell cycle, and down-regulates the expression of cyclin B1 which is involved in cell progression from G2 phase to mitosis, and down-regulate cyclin E which is involved in the progression of cells from G0/G1 phase to the S phase, thereby to induce cell cycle arrest.

Further, the expression enhancement of cell cycle-related genes was investigated using a LoVo cell line treated with FA-FOS I at a concentration of 0.198 μg/ml for 24 hr. Gene expression levels were measured using Qiagen's RT2 Prolifer PCR Array Human Cell cycle. Table 6 below shows the profiling results of genes expressed in relation to the cell cycle in LoVo cell lines treated with FA-FOS I. As shown in the following Table 6, it was observed that there are 9 major upregulation genes and 4 major downregulation genes directly involved in cell cycle progression and proliferation.

TABLE 6

| | Fold Regulation | | Fold Regulation |
|---|---|---|---|
| | (compared to | | (compared to |
| | FA-FOS I | | FA-FOS I |
| gene | non-treatment) | gene | non-treatment) |
| BRCA1 | 1.67 | CDK6 | 1.54 |
| BCL2 | 1.61 | CDKN2B | 1.40 |
| BRCA2 | 2.09 | CDK5RAP1 | 1.41 |
| ATM | 1.39 | MCM4 | −1.13 |
| ATR | 1.63 | SERATD1 | −1.13 |
| BCCIP | 1.40 | CDC34 | −1.03 |
| CCNG2 | 1.48 | E2F1 | −1.08 |
| CCNT1 | 1.66 | | |

5. Anticancer Activity of Ferulic Acid-Fructooligosaccharide Conjugate in In-Vivo Experiment

(1) Anticancer Activity of Ferulic Acid-Fructooligosaccharide Conjugate in a Mouse Model in which Colon Carcinoma is Induced with AOM-DSS

The anticancer activity of FA-FOS I and FA-FOS II was evaluated using a mouse model in which colon carcinoma was induced with AOM (Azoxymethane)-DSS (Dextran Sodium Sulfate). 5 to 6 weeks aged C57BL/6 female mice were randomly assigned to the following 6 groups, each of 7 mice, and the necessary treatments were performed for each group.

(i) Naive

(ii) Control: a mouse in which colon carcinoma was induced with AOM-DSS and to which no separate drug was administered.

(iii) Oxaliplatin treated group (Oxaliplatin): A drug is prepared by dissolving oxaliplatin in water, and the drug is orally administered to mice at a dose of 30 mg/kg (b.w) based on the amount of oxaliplatin.

(iv) Ferulic acid treated group (FA): A drug is prepared by dissolving ferulic acid in water and the drug is administered orally to mice at a dose of 100 mg/kg (b.w) based on the amount of ferulic acid.

(v) FA-FOS I treated group (FA FOS-I): the ferulic acid-fructooligosaccharide conjugate FA-FOS I is suspended in water to prepare a drug, and the drug is administered orally to mice at a dose of 500 mg/kg (b.w) based on the amount of FA-FOS I.

(vi) FA-FOS II treated group (FA FOS-II): the ferulic acid-fructooligosaccharide conjugate FA-FOS II is dissolved in water to prepare a drug, and the drug is administered orally to mice at a dose of 3890 mg/kg (b.w) based on the amount of FA-FOS II.

For reference, when comparing the ferulic acid-fructooligosaccharide conjugates FA-FOS I and FA-FOS II with the amount of the ferulic acid, each of 500 mg of FA-FOS I and 3890 mg of FA-FOS II corresponds to 100 mg of ferulic acid.

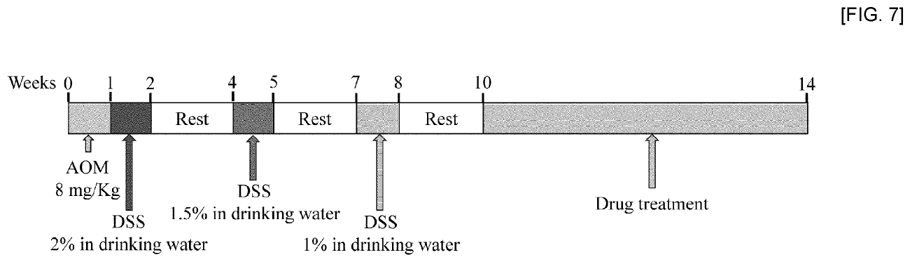

FIG. 7 schematically shows the experimental process to evaluate the anticancer activity of ferulic acid-fructooligosaccharide conjugate using a mouse model in which colon carcinoma is induced using AOM-DSS.

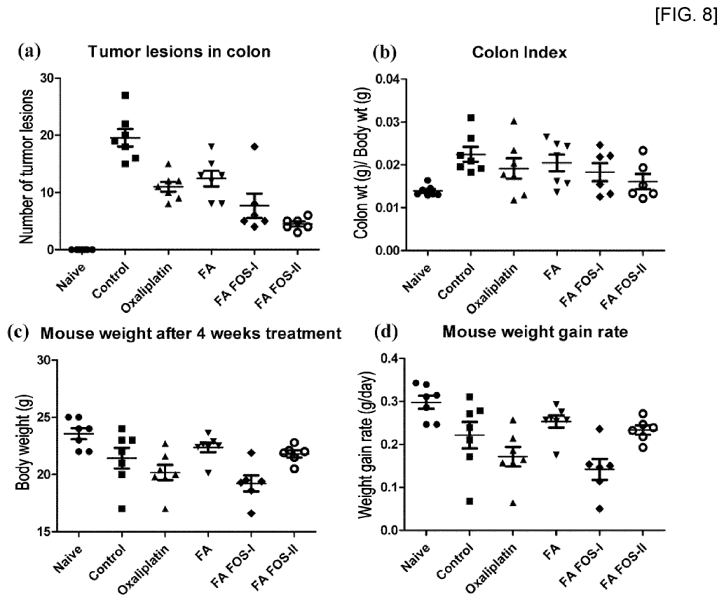

FIG. 8 shows the experimental results of evaluating the anticancer activity of ferulic acid-fructooligosaccharide conjugate using a mouse model in which colon carcinoma was induced using AOM-DSS. (a) the total number of tumor lesions in the colon, (b) colon index, (c) the body weight of the mouse at the end of the drug treatment and (d) the weight gain of the mouse during the treatment regimen for 4 weeks. As shown in FIG. 8, the number of tumor lesions in the FA-FOS II treated group was reduced by 77.1% (p<0.001) compared to that of control. Further, FA-FOS II treated group showed higher tumor reduction compared to oxaliplatin treated group and ferulic acid treated group. Further, there was no significant difference in mouse colon index between FA-FOS II treated group and naive.

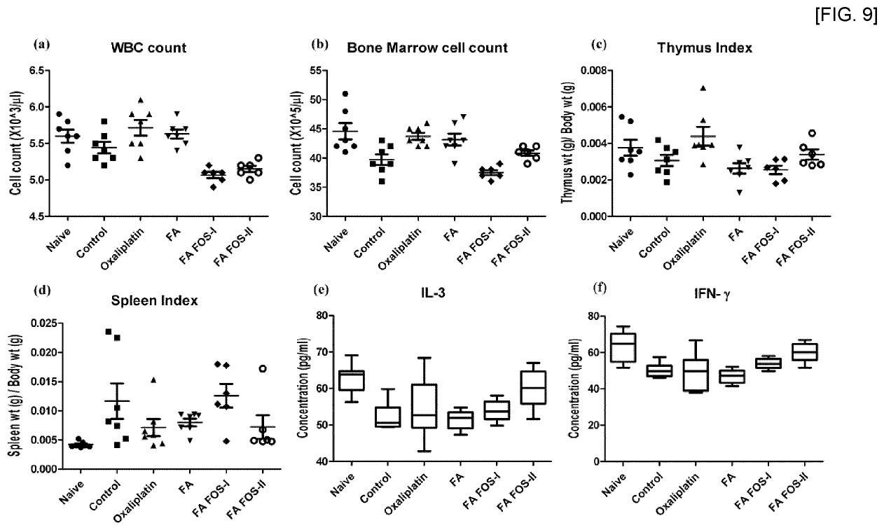

FIG. 9 shows the results of immunological parameters among experimental results that evaluated the anticancer activity of ferulic acid-fructooligosaccharide conjugate using a mouse model in which colon carcinoma is induced using AOM-DSS. In FIG. 9, (a) is the number of white blood cells (WBCs) in the whole blood, (b) is the total number of bone marrow cells, (c) is the thymus index (thymus/weight), and (d) is the spleen index (spleen/weight), (e) is the in-plasma interleukin 3 concentration, and (f) is the in-plasma interferon gamma concentration. As shown in FIG. 9, the number of WBCs among the immune-related parameters decreased by about 6.90% in the FA-FOS I treated group compared to the control (p<0.01). The number of WBCs among the immune-related parameters decreased by about 5.38% in the FA-FOS II treated group compared to the control (p<0.05). However, when considering the number of bone marrow cells, thymus index and spleen index, etc., there is no immune suppression induced with FA-FOS I treatment and FA-FOS II treatment. On the other hand, the IL-3 concentration in the plasma of mice increased by about 15% in the FA-FOS II treated group compared to the control (p<0.05). This result suggests that there is stimulation/sensitization of T cells to tumor antigens due to the FA-FOS II treatment.

(2) In Vivo Pharmacokinetics of FA-FOS II in Rats

In order to evaluate the in vivo pharmacokinetics of FA-FOS II, the release profile of ferulic acid (FA) released from FA-FOS II in the colon of rats was investigated. Specifically, male Sprague Dawley rats weighing 180 to 250 g were randomly assigned to three groups as follows.

(i) Naive: No separate drug is administered thereto

(ii) Ferulic acid (FA) administered group: a drug is prepared by dissolving ferulic acid (FA) in water and is administered orally thereto at a predetermined dose

(iii) FA-FOS II administered group: a drug is prepared by dissolving FA-FOS II in water and is administered orally thereto at a predetermined dose

Ferulic acid (FA) was orally administered to the rats at a dose of 100 mg/kg body weight, based on the amount of ferulic acid (FA), in the state dissolved in sterile water. Further, FA-FOS II was orally administered thereto at a dose of 3.89 g/kg body weight (equivalent to 100 mg/kg body weight in terms of the amount of ferulic acid) based on the amount of FA-FOS II, in the state dissolved in sterile water. After single dose administration via oral intake, rat blood was continuously sampled over 0, 0.5, 1, 3, 6, 8, 12, 24 hr. The sampled samples were subjected to LC MS/MS analysis in a multiple reaction monitoring (MRM) manner of a molecular weight 193.0/133.9 Da (intact mass/fragment mass of FA) to quantify the FA released from plasma.

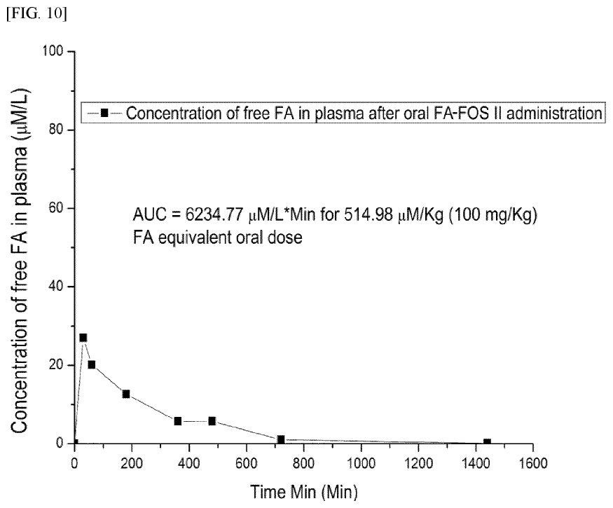

FIG. 10 shows the pharmacokinetic release profile of ferulic acid (FA) released from FA-FOS II in rat plasma. As shown in FIG. 10, the Tmax of FA released from FA-FOS II in the FA-FOS II administered group was about 30 minutes. On the other hand, in the ferulic acid (FA) administered group, the Tmax was about 5 minutes. Further, as shown in FIG. 10, the average retention time of FA released from FA-FOS II in the FA-FOS II administered group is about 240 minutes. In the ferulic acid (FA) administered group, T_{1/2} is about 30 minutes. Thus, the former time was a relatively long time. The Cmax of free FA observed after the FA-FOS II administration was 27.08 μM. The AUC (Area Under Curve) indicates the degree of exposure of the drug molecule to the body (spread of the drug molecule in the plasma). In the FA-FOS II administered group, the AUC (Area Under Curve) was 6234.77 μM*Min/L. This is about 2.02 times higher than the AUC in the group administered with the same dose of ferulic acid (FA). Therefore, it may be seen that when FA-FOS II is administered thereto, the bioavailability of ferulic acid (FA) molecules is about twice that in the administration of ferulic acid (FA) itself.

(3) Anticancer Activity of FA-FOS II in a Xenograft Mouse Model with HT-29 Tumor (Human Tumor)

The anticancer activity of FA-FOS II was evaluated using xenograft mice bearing HT-29 tumors. Specifically, BALB/c nude mice were randomly assigned to the following four groups, and necessary treatments were performed for each group. Further, a xenograft mouse model bearing HT-29 tumor was produced by the following method. First, the HT-29 cell line was cultured and harvested in RPMI 1640 medium with 10% FBS in 5% CO₂ condition. The harvested HT-29 cell line culture was diluted and injected into the right lateral subcutaneous area of BALB/c nude mice in an amount of 1×10⁷ cells/0.2 ml/mice, and then we waited until the tumor volume reached 150 to 200 mm³.

(i) Naive

(ii) Control: xenograft mice bearing HT-29 tumors which are not subjected to administration of separate drugs

(iii) 5-fluorouracil treated group (5-Fu): We prepares a drug by dissolving 5-fluorouracil in 0.9% saline, and administers the drug in an intraperitoneal IP manner at a dose 0.2 ml or less for 5 consecutive days for 1 week (once a day), takes a break for a week, and then administers the drug in the IP manner twice a week for 2 weeks at a dose of 15 mg/kg (b.w) per administration, based on the amount of 5-fluorouracil (this particular treatment regimen was selected due to the side effects of 5-FU as observed by the investigator).

(iv) FA-FOS II treated group (FA FOS II): ferulic acid-fructooligosaccharide conjugate FA-FOS II is dissolved in distilled water to prepare a drug. The drug is orally administered daily for 4 weeks at a dose of 3890 mg/kg (b.w) per administration, based on the amount of FA-FOS II.

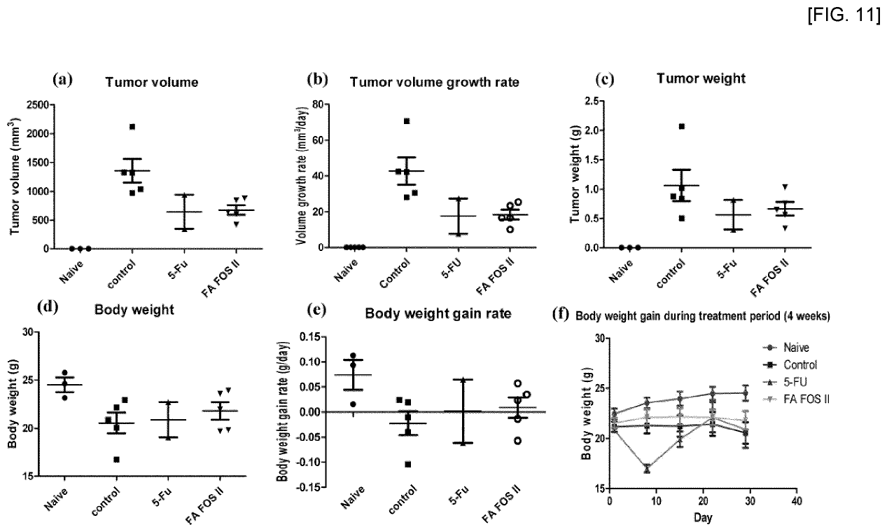

FIG. 11 shows the results of experiments evaluating the anticancer activity of ferulic acid-fructooligosaccharide conjugate FA-FOS II using the HT-29 tumor bearing xenograft mouse model. (a) Tumor volume after 4 weeks of treatment, (b) Tumor growth rate, (c) tumor weight after 4 weeks of treatment, (d) body weight after 4 weeks of treatment, (e) daily weight gain during the course of treatment, and (f) weight increase profile during treatment. As shown in FIG. 11, the tumor volume in the FA-FOS II treated group decreased by about 50% compared to the control (p<0.05). Further, in the FA-FOS II treated group, the average tumor weight decreased by about 37.34% compared to the control. The body weight measured to indirectly determine the toxicity or side effects of drug treatment increased by about 6% in the FA-FOS II treated group compared to the control. Further, the daily weight gain rate increased by about 41.47% in the FA-FOS II treated group compared to the control. During the entire treatment period, the weight gain in the FA-FOS II treated group was higher than that of the control. This result suggests that ferulic acid-fructooligosaccharide conjugate FA-FOS II does not cause side effects.

6. Antioxidant Activity of Ferulic Acid-Fructooligosaccharide Conjugate

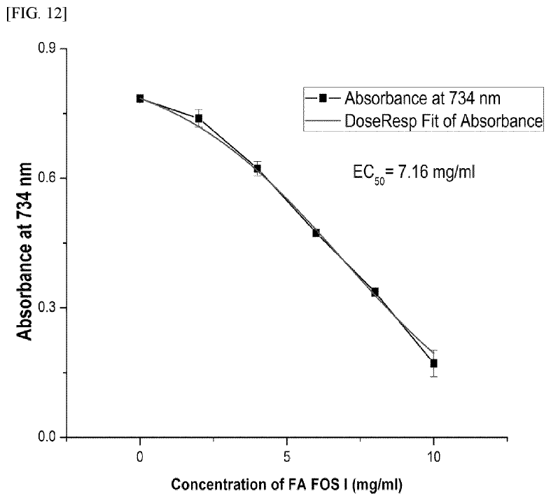

The antioxidant activity of FA-FOS I was measured using ABTS[2,2′-azino-bis(3-ethylbenzothiazoline-6-sulphonic acid)] cation radical scavenging ability assay. ABTS cationic radical scavenging ability assay was performed according to Arda Serpen et al., 2007. Specifically, after adding lyophilized FA-FOS I, in various amounts, to an eppendorf tube, 1.7 ml of an ABTS cation radical reagent as chemically pre-generated using MnO₂ was added thereto, followed by stirring for 2 minutes such that the mixture was uniformly mixed and the reaction proceeded for 6 minutes. Thereafter, the reaction mixture was centrifuged to collect the supernatant, and the absorbance of the supernatant was measured at 734 nm using a UV-VIS plate reader. FIG. 12 shows the results of measuring the antioxidant activity of FA-FOS I using ABTS[2,2′-azino-bis(3-ethylbenzothiazoline-6-sulphonic acid)] cation radical scavenging ability assay. In FIG. 12, ‘FA FOS I’ represents FA-FOS I. Further, as shown in FIG. 12, FA-FOS I showed excellent antioxidant activity and the EC₅₀ of FA-FOS I was about 7.16 mg.

As described above, the present disclosure has been described based on examples, but the present disclosure is not necessarily limited thereto. It goes without saying that various modifications thereto may be implemented without departing from the scope and idea of the present disclosure. Therefore, the scope of protection of the present disclosure should be construed as including all embodiments falling within the scope of the claims attached to the present disclosure.

1. A phytochemical-fructooligosaccharide conjugate selected from compounds represented by a following chemical formula II:
PhA-Glu-(Fru)ₙ-Fru [Chemical formula II]
wherein in the chemical formula II, ‘PhA’ represents a phytochemical in a form of a phenolic acid, ‘Glu’ represents glucose, and ‘Fru’ represents fructose;
wherein ‘PhA’ and ‘Glu’ are connected to each other via an ester bond, ‘Glu’ and ‘Fru’ are connected to each other via a glycosidic bond, and ‘Fru’ and ‘Fru’ are connected to each other via a glycosidic bond;
wherein n is a number of ‘Fru’s connected to each other via a glycosidic bond and is selected from an integer of 1 to 59.

2. The phytochemical-fructooligosaccharide conjugate of claim 1, wherein the phytochemical in the form of phenolic acid is selected from ferulic acid, caffeic acid, cinnamic acid, chlorogenic acid, coumarin, cinapinic acid, cichoric acid, diferulic acid, coumaric acid, salicylic acid, 3-hydroxybenzoic acid, 4-hydroxybenzoic acid, vanillic acid, gallic acid or ellagic acid.

3. The phytochemical-fructooligosaccharide conjugate of claim 1, wherein the phytochemical in the form of phenolic acid is connected to the fructooligosaccharide via an ester bond between a hydroxyl group at carbon 6 of glucose of the fructooligosaccharide and a carboxyl group present at the phytochemical.

4. The phytochemical-fructooligosaccharide conjugate of claim 1, wherein the phytochemical-fructooligosaccharide conjugate is selected from ferulic acid-fructooligosaccharide conjugates represented by a following chemical formula IV: 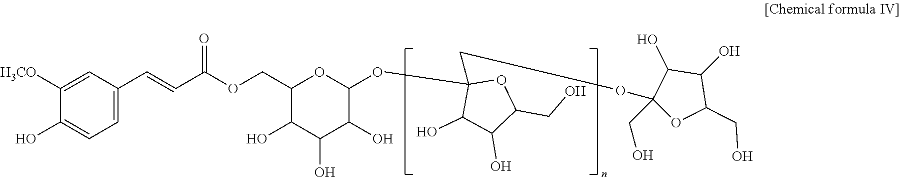
wherein in the chemical formula IV, n is selected from an integer of 1 to 59.

5. The phytochemical-fructooligosaccharide conjugate of claim 4, wherein the phytochemical-fructooligosaccharide conjugate is composed of or contains a ferulic acid-fructooligosaccharide conjugate represented by a following chemical formula VI as a main component:

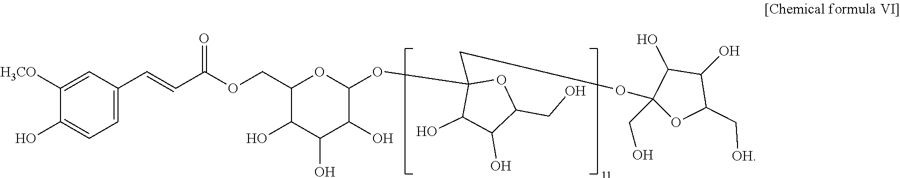

6. A pharmaceutically acceptable salt of the phytochemical-fructooligosaccharide conjugate of claim 1.

7. A method for producing a phytochemical-fructooligosaccharide conjugate, the method comprising:
(a) adding, dissolving, and heating a phytochemical in a form of phenolic acid and a catalyst for an esterification reaction to a reaction solvent to perform an activation reaction of the phytochemical, and thus obtaining a first reaction mixture containing a phytochemical in an activated form; and
(b) adding a fructooligosaccharide represented by a following general structural formula to the first reaction mixture, and heating the mixture under an inert gas atmosphere to perform an esterification reaction between the fructooligosaccharide and the phytochemical, and thus obtaining a second reaction mixture containing the phytochemical-fructooligosaccharide conjugate of claim 1,
[General Structural Formula of Fructooligosaccharide]
Glu-(Fru)ₖ
wherein in the general structural formula of the fructooligosaccharide, ‘Glu’ denotes glucose, ‘Fru’ denotes fructose, and k denotes a number of fructoses connected to each other via a glycosidic bond, and is selected from an integer of 2 to 60.

8. The method of claim 7, further comprising (c) cooling and leaving the second reaction mixture to precipitate the phytochemical-fructooligosaccharide conjugate, and performing centrifugation and washing sequentially to obtain a purified phytochemical-fructooligosaccharide conjugate.

9. The method of claim 7, wherein the phytochemical activation reaction in the (a) is performed at 45 to 120° C. for 2 to 20 hr.

10. The method of claim 7, wherein the esterification reaction in the (b) is performed at 70 to 150° C. for 2 to 15 hr.

11. The method of claim 7, wherein the catalyst for the esterification reaction is carbonyldiimidazole (N,N′-carbonyldiimidazole, CDI).

12. The method of claim 7, wherein a molar ratio of the phytochemical in the form of the phenolic acid, the catalyst for esterification, and the fructooligosaccharide used for producing the phytochemical-fructooligosaccharide conjugate is selected from a range of 2:2:1 to 10:10:1.

13. An anticancer composition comprising the phytochemical-fructooligosaccharide conjugate of claim 4 or a pharmaceutically acceptable salt thereof as an active ingredient.

14. A method for preventing or treating a colorectal cancer comprising, administering the composition comprising the phytochemical-fructooligosaccharide conjugate of claim 4 or a pharmaceutically acceptable salt thereof as an active ingredient to a subject in need thereof.

15. A method for inhibiting colorectal cancer metastasis comprising, administering the composition comprising the phytochemical-fructooligosaccharide conjugate of claim 4 or a pharmaceutically acceptable salt thereof as an active ingredient to a subject in need thereof.
